(19) United States
(12) Patent Application Publication
(10) Pub. No.: US 2024/0246056 A1
(43) Pub. Date: Jul. 25, 2024

(54) MANUFACTURING DEVICE FOR A PHARMACEUTICAL PRODUCT

(71) Applicants: CureVac RNA Printer GmbH, Tübingen (DE); Tesla Automation GmbH, Prüm (DE)

(72) Inventors: Benyamin YAZDAN PANAH, Tübingen (DE); Tilmann ROOS, Tübingen (DE); Telmo GRACA, Tübingen (DE); Felix BERTSCH, Tübingen (DE); Veronika FONTIUS, Tübingen (DE); Michael RAUEN, Prüm (DE); Nico SCHOLTES, Prüm (DE); Philipp HOFFMANN, Prüm (DE); Martin KUNZE, Tübingen (DE)

(73) Assignees: CureVac RNA Printer GmbH, Tübingen (DE); Tesla Automation GmbH, Prüm (DE)

(21) Appl. No.: 18/043,577

(22) PCT Filed: Sep. 1, 2021
(86) PCT No.: PCT/EP2021/074070

§ 371 Date: Feb. 28, 2023

(30) Foreign Application Priority Data
Sep. 1, 2020 (WO) PCT/EP2020/074375

(51) Int. Cl. B01J 19/00

(52) U.S. Cl. CPC B01J 19/0046; B01J 2219/00423; B01J 2219/00495; B01J 2219/00716; B01J 2219/00722

(57) ABSTRACT

The present invention relates to a manufacturing device for a pharmaceutical product, a manufacturing module for a pharmaceutical product, a manufacturing system for a pharmaceutical product, a method for producing a pharmaceutical product, a method for producing a DNA template, a method for producing an RNA, a method for producing a formulated RNA and uses of the manufacturing device, module or system. The manufacturing device for a pharmaceutical product comprises a housing, a process chamber, a technical chamber, a separation element, and a control unit. The housing is closed relative to the environment outside the housing. The housing encompasses the process chamber and the technical chamber. The process chamber is separated from the technical chamber by the separation element. The control unit is configured to control a gas flow through the process chamber. The control unit is configured to control the gas flow to provide in the process chamber a positive pressure relative to the environment. The control unit is further configured to control the gas flow to provide the gas flow through the process chamber as a gas shower falling in the direction of gravity.

This application is a national phase application under 35 U.S.C. § 371 of International Application No. PCT/EP2021/074070, filed Sep. 1, 2021, the entire contents of which are hereby incorporated by reference. International Application No. PCT/EP2021/074070 claims benefit of International Application No. PCT/EP2020/074375, filed Sep. 1, 2020.

### FIELD OF THE INVENTION

The present invention relates to a manufacturing device; a manufacturing module comprising at least two manufacturing devices; a manufacturing system comprising a manufacturing device or a manufacturing module and a clean room; a method for producing DNA, a method for producing a DNA template; a method for producing RNA, preferably mRNA; a method for producing formulated RNA, preferably LNP-formulated RNA; a use of the manufacturing device, the manufacturing module or the manufacturing system for the production of a pharmaceutical product; a use of the manufacturing device, the manufacturing module or the manufacturing system for the production of DNA; a use of the manufacturing device, the manufacturing module or the manufacturing system for the production of a DNA template; a use of the manufacturing device, the manufacturing module or the manufacturing system for the production of RNA, preferably mRNA; and a use of the manufacturing device, the manufacturing module or the manufacturing system for the production of formulated RNA, preferably LNP-formulated RNA.

### BACKGROUND OF THE INVENTION

Active pharmaceutical ingredients (APIs) in the form of biomolecules are used today largely in therapies of a variety of diseases. Such biomolecules are e.g. therapeutic antibodies, peptides and nucleic acids. Therapeutic nucleic acids including RNA molecules represent an emerging class of drugs. RNA-based therapeutics include mRNA molecules encoding antigens for use as vaccines (Fotin-Mleczek et al. 2012. J. Gene Med. 14(6):428-439). In addition, it is envisioned to use RNA molecules for replacement therapies, e.g. providing missing proteins such as growth factors or enzymes to patients (Karikó et al., 2012. Mol. Ther. 20(5):948-953; Kormann et al., 2012. Nat. Biotechnol. 29(2):154-157). Furthermore, the therapeutic use of noncoding immunostimulatory RNA molecules (e.g. WO2009/095226A2) and other noncoding RNAs such as microRNAs and long noncoding RNAs (Esteller, 2011. Nat. Rev. Genet. 12(12):861-74) or RNAs suitable for genome editing (e.g. CRISPR/Cas9 guide RNAs) is considered.

Accordingly, RNA-based therapeutics with the use in immunotherapy, gene therapy and vaccination belong to the most promising and quickly developing therapeutic fields in modern medicine.

Currently established manufacturing processes for APIs in the form of biomolecules approved by regulatory authorities typically implement many separate manufacturing steps, all of which must comply with GMP-standards.

Such steps are typically carried out in GMP-compliant rooms that are present in production plants and thus not portable. Furthermore, the rooms are typically set up for the production of a specific biomolecule and are thus not flexible but need to be rebuilt to a rather large extent if a different biomolecule needs to be produced. In summary, the manufacturing of biomolecules at API-grade requires a large degree of manual handling in a GMP-regulated, rather inflexible and fixed laboratory, executed by well-trained technical staff. In consequence, current established manufacturing processes are time consuming, cost intensive, and require a lot of laboratory space and laboratory equipment.

Even if such manufacturing processes are volume tailorable as disclosed in WO 2019/096925, such volume tailorable systems comprise one or more multi-product suites that comprise entrances, offices as well as storages; support areas, media preparation areas and buffer preparation areas; entry corridors to the production area; and the production area; as well as exit corridors, etc. (see in particular FIG. 5 of WO 2019/096925). Furthermore, WO 2019/096925 discloses a unidirectional flow in the one or more multi-product suites in terms of personnel, raw material, products and waste in order to comply with the biosafety level classification 2. Reference to a circulation system is made in WO 2019/096925 such that this system maintains the unidirectional flow (of e.g. personnel) while making two or more suites interconnectable, wherein the (initially sealed) connection may comprise e.g. a corridor after two suites have been connected and the seal has been broken (see in particular FIGS. 2a and 5 of WO 2019/096925). Accordingly, WO2019/096925 does not disclose manufacturing devices, e.g. in the form of rather small portable machines or automats, configured to produce APIs under (c)GMP-compliant conditions.

### SUMMARY OF THE INVENTION

As outlined above, there is the problem associated with common manufacturing processes for biomolecules as APIs that the processes are rather inflexible, not portable and typically require a large degree of manual handling of well-trained technical staff. Thus, there is a need for providing an improved manufacturing device, which is more flexible. The manufacturing device should in particular provide the benefits to be operated under GMP-compliant conditions, to be portable and/or to be automated. Such a manufacturing device would reduce the time for manufacturing the API (e.g. of a vaccine) tremendously which is particularly important in the context of an outbreak of a virus (e.g. pandemic outbreak or local outbreak). Such a portable API manufacturing device could be easily shipped, installed and operated in pandemic hotspot regions around the globe and would therefore allow for a rapid production of a vaccine in said region. In essence, after having been shipped and installed in a pandemic hotspot region, the production of a vaccine (in particular an mRNA-based vaccine) could immediately start after the genetic information of the virus is known, i.e. after the DNA or RNA of the virus has been sequenced. A first step could then e.g. relate to the de-novo production of DNA comprising the viral sequence, followed e.g. by the production of template DNA, further followed by the production of mRNA by in vitro transcription using this template DNA, preferably further followed by formulating the mRNA, e.g. in lipid nanoparticles. The formulated RNA may then be filled and packaged, and provided in the form of a pharmaceutical product, namely as vaccine, in particular to the subjects in the pandemic hotspot region. Therefore, it is of paramount importance that the manufacturing device of the invention is configured to produce APIs under (c)GMP-compliant conditions.

The above problem is solved by the subject-matter of the independent claims, wherein further embodiments thereof are provided in the dependent claims.

In a first aspect, the present invention is directed to a manufacturing device for a pharmaceutical product. The manufacturing device comprises a housing, a process chamber, a technical chamber, a separation element, and a control unit. The housing is closed relative to the environment outside the housing. The housing encompasses the process chamber and the technical chamber. The process chamber is separated from the technical chamber by the separation element. The control unit is configured to control a gas flow through the process chamber. The control unit is configured to control the gas flow to provide in the process chamber a positive pressure relative to the environment. The control unit is further configured to control the gas flow to provide the gas flow through the process chamber as a gas shower falling in the direction of gravity.

As outlined below, a pharmaceutical product may in particular be RNA, more particularly an RNA vaccine. Thus, for example, the manufacturing device according to the present invention may be used for a flexible and fast vaccine production, particularly a flexible and fast RNA vaccine production. The present manufacturing device may be used in particular for mRNA-based vaccines e.g. during infectious disease epidemics and pandemics as well as for diverse cancer diseases including personalized therapies.

The present manufacturing device can be understood as a closed housing with different chambers for different purposes.

The process chamber may be suitable and used for manufacturing the pharmaceutical product. The process chamber may also be referred to as “wet part” of the manufacturing device in a sense that essentially all wet processes during the production process take place in the process chamber. “Wet processes” means e.g. the provision of buffers and/or reactants and/or products to and from vessels or to and from chromatographic columns, the collection of waste fluid or waste gases, etc., as well as any connections of such buffers and/or reactions and/or products (e.g. connections in the form of flexible tubes). Such wet processes should ideally be separated from any technical media supply (in particular electric supply such as power cables) in order to render impossible or at least minimize accidents due to leakage of liquids in the process chamber and possible consequences thereof, in particular due to voltage. Should accidents due to leakage occur in the process chamber, safety is improved as there is the afore-mentioned separation, and it will only be required to clean the process chamber. In preferred embodiments, the process chamber is not a room or a laboratory of a building.

As outlined below for the control unit, once in operation, there will be a positive pressure in the process chamber as well as a gas shower falling in the direction of gravity (i.e. a constant unidirectional flow in the direction of gravity) in the process chamber. This provides for suitable conditions to manufacture a pharmaceutical product in the process chamber, which may in view of this also be referred to as “clean part” of the manufacturing device.

The technical chamber may be suitable and used for housing apparatus, as e.g. a pump, a motor, a mixer, a processor or the like, which are not in direct contact with the process media. The technical chamber is typically suitable and used for providing (e.g. housing) technical media supply, which is in particular power supply, e.g. a power supply for an apparatus/unit that is located in the process chamber and that is in direct contact with the process media. The technical chamber may also be referred to as “dry part” of the manufacturing device in a sense that essentially no wet processes as defined above take place in the technical chamber. As noted above, this inter alia results in increased safety. One may refer to the technical chamber as being “not as clean” as the process chamber since the required level of cleanliness in the technical chamber is lower than in the process chamber. In preferred embodiments, the technical chamber is not a room or a laboratory of a building.

The afore-mentioned two chambers are separated by the separation element, which can be understood as a plate. The separation element is preferably plate-shaped, in particular plate-shaped when looking at the separation element from the process chamber. Thus, as will be outlined below, in order not to disturb the gas flow, the separation element may comprise at least one appendix with an opening towards the process chamber and an extension in the direction of the technical chamber, where apparatus may be housed that are in contact with the process media (see FIG. 5b). In an embodiment, the separation element is not a corridor, in particular not an entry corridor. In an embodiment, the separation element is not a chamber. In an embodiment, the separation element is not a wall of a room or a laboratory of a building. In yet another embodiment, the separation element is plate-shaped and not a corridor. In still another embodiment, the separation element is plate-shaped and not a chamber. In an embodiment, the separation element is plate-shaped and not a wall of a room or a laboratory of a building.

The control unit can be understood as any control device for a gas flow, as e.g. a valve or a processor. The gas flow may be provided by a flow unit. The flow unit may be at least a pump or a HVAC system, and may also be referred to as fan-system. The flow unit may be arranged outside the housing (external flow unit) or preferably within the housing (internal flow unit). The flow unit preferably comprises a filter unit (which may be referred to as “fan filter unit” and which preferably comprises a HEPA filter as described below). The flow unit may be configured to provide, preferably variable, volumes of gas. The control unit is configured to control the gas flow through the process chamber and configured to control the gas flow to provide a positive pressure in the process chamber as well as a gas shower through the process chamber. In other words, the control unit may be configured to adjust different pressure in the gas flow. The gas may be clean air, air with additives, an inert gas or any other gas. The positive pressure in the process chamber can be understood as overpressure relative to the environment outside the housing and/or a higher pressure than in the technical chamber. The gas shower may fall from a higher level in the process chamber to a lower level in the process chamber. The positive pressure and the gas shower in the process chamber may protect open process steps from particulate contamination.

A positive pressure in the process chamber as well as a gas shower falling in the direction of gravity (i.e. a constant unidirectional flow in the direction of gravity) in the process chamber are essential elements for providing suitable conditions to manufacture a pharmaceutical product in the process chamber. Importantly, such conditions allow for the production under the required standards, namely in ensuring that no air from the outside (which is optionally contaminated or polluted) can enter the process chamber due to the positive pressure and the unidirectional flow (such a flow is e.g. also known from laminar flow hoods in order to protect the working environment of the hood).

Accordingly, the control unit and the (different) pressures and flows achieved thereby are essential elements of the present invention.

### Description of the Manufacturing Device

The present manufacturing device may provide a closed (bio-synthetic) process from raw materials to a final product. Of course, corresponding waste materials typically also accumulate, which are collected and disposed of as applicable. Preferably, the “closed (bio-synthetic) process” is performed inside the closed manufacturing device, wherein the process is controlled or supervised by a human operator located outside of the manufacturing device (see e.g. FIG. 2).

The present manufacturing device can be understood as manufacturing-in-a-box. It may provide the following advantages:

The present manufacturing device is a very flexible and versatile platform. It can be adapted to various purposes and uses. It allows for a modular manufacturing or production of pharmaceutical products. It may be understood as a single entity suitable for modular manufacturing, which means that it can be adapted to various purposes. The single entity (i.e. a single manufacturing device according to the present invention) can be sufficient for a specific manufacturing method or several entities can be connected (resulting in a manufacturing module according to the present invention) to perform a specific manufacturing method, e.g. in the form of a manufacturing line or chain. Such modular design allows for an easy handling, process improvements and/or maintenance procedures. The technologies and operations used within the present manufacturing device as a module as well as the choice of product-contacting materials can be pre-evaluated before its actual use to provide a high quality manufacturing of pharmaceutical products. As a result, the present manufacturing device allows for a very robust and reproducible manufacturing of the pharmaceutical product. Further, the present manufacturing device may allow a (fully or partially) automated manufacturing of a pharmaceutical product.

The present manufacturing device can be dimensioned to be very small. It may have a low footprint and form a low footprint-manufacturing entity. The footprint may be in a range of about 100×about 100×about 200 cm such that it can easily e.g. shipped and stored in a typical shipping container of the usual size. The present manufacturing device is thus portable to allow transportation to e.g. regions of an outbreak of a pandemic. It thereby generally allows a local, decentral production of a pharmaceutical product (e.g. a vaccine).

The present manufacturing device generally allows for a very fast manufacturing of pharmaceutical products. A change and preparation of the manufacturing device to a specific pharmaceutical product as well as the manufacturing of the specific pharmaceutical product itself may be very fast. There may be no need for adjustment or calibration steps. There may be no or fewer need for cleaning steps. The turnover time for e.g. a vaccine manufacturing may be less than about a week.

The present manufacturing device is generally very easy to clean, which allows reducing the cleaning efforts. The reason is a sanitary and hygienic design right from the beginning. It may comprise an aluminium housing, wherein the aluminium may have been pre-treated with e.g. an anodic oxidation and passivation, an anti-microbial surface finish with e.g. silver ion containing colours, electro polished surfaces, a use of compatible materials throughout the manufacturing device, accessibility of many or every part of the manufacturing device for cleaning, smooth, seamless and cleanable exposed surfaces, harmonized inner diameters and geometries for optimal automated cleaning in place (CIP) procedures and/or anti-microbiological cleansing, a sealing of any crevices or hollow areas, a reduction or stop of any build-up of condensation and precipitation, etc. In particular, the housing, the chamber(s) and/or the separation element may be made of aluminium, preferably surface-treated aluminium to allow for CIP. Additionally or alternatively, surfaces outside and/or within the housing and in particular within the chamber(s) may have an anti-microbial surface finish. Further, apparatus within the housing and in particular within the process chamber may be encapsulated to provide a smooth surface. “Apparatus” may be understood as a pump, a motor, a mixer, a processor or the like. Further, moving parts or parts directing technical media may be encapsulated to be safe and to allow an easy cleaning. Additionally or alternatively, apparatus within the housing and in particular within the process chamber may be embedded into a wall and in particular into the separation element to be, in best case, flush with the respective wall or surface. The housing or chamber wall as well as the separation element may be provided with an appendix to house the apparatus as flush as possible. These options improve the cleanability as well as maintain the gas flow without any disruptions or undesired circumventions due to non-flush elements in the process chamber.

The present manufacturing device can be operated under GMP (guidelines for good manufacturing practice) —compliant conditions. In an embodiment, the GMP requirements are the requirements of the EU Guidelines to Good Manufacturing Practice Medicinal Products for Human and Veterinary Use, Annex 1, Manufacture of Sterile Medicinal Products (corrected version), European Commission, Brussels, 25 Nov. 2008 (revised). In an embodiment, the GMP requirements are the requirements of the FDA (2004) Guidance for Industry, Sterile Drug Products Produced by Aseptic Processing—Current Good Manufacturing Practice, U.S. Department of Health and Human Services, Food and Drug Administration, Center for Drug Evaluation and Research (CDER) Center for Biologics Evaluation and Research (CBER) Office of Regulatory Affairs (ORA) Pharmaceutical CGMPs. In an embodiment, the present manufacturing device may be operated compliant to CE, ISO or GAMP5 Standards. The present manufacturing device may allow a GMP compliant manufacturing-in-a-box. GMP compliant conditions may comprise compliant machine controls and data logging, as for example automatic logging and reporting of critical process parameters and/or quality attributes, e.g. pressure, humidity, temperature, UV-absorption, total organic carbon, infrared absorption spectra, flow, power consumption, an error causing event (opened door, leakage, shortcut, pressure loss . . . ) and the like.

### Embodiments Relating to the Manufacturing Device

In an embodiment, the housing is sealed relative to the environment. In the same or another embodiment, the process chamber is sealed relative to the technical chamber. Throughout this application, the term “sealed” can be understood as compliant with at least IP64. IP codes are based on Degrees of protection provided by enclosures (IP Code) (IEC 60529:1989+A1:1999+A2:2013); German version EN 60529:1991+A1:2000+A2:2013 and/or ISO 20653:2013-02 Road vehicles—Degrees of protection (IP code)—Protection of electrical equipment against foreign objects, water and access. The term “sealed” can be understood as dust-tight and protection against splashing of water. “Dust-tight” can be understood as no ingress of dust and complete protection against contact. For testing, a vacuum is typically applied, test duration of up to 8 hours based on airflow, details can be found in above cited standards. “Protection against splashing of water” can be understood as water splashing against an enclosure from any direction shall have no harmful effect, utilizing either an oscillating fixture for 10 minutes or a spray nozzle with no shield for 5 minutes minimum, details can be found in above cited standards. A sealed housing or a sealed process chamber may have the benefit to be in particular clean, which means to have and to maintain low levels of particulates, such as dust, airborne organisms, or vaporized particles.

In an embodiment, the process chamber is configured to be at least a grade D room according to the above mentioned EU guidelines for good manufacturing practice for medicinal products, annex 1. Preferably, the process chamber is configured to be at least a grade C room. More preferably, the process chamber is configured to be at least a grade B room. Even more preferably, the process chamber is configured to be a grade A room. Grade A to D rooms may provide the benefit and are designed to be in particular clean, which means to have and to maintain extremely low levels of particulates, such as dust, airborne organisms, or vaporized particles.

Cleanliness may be quantified by a number of particles per cubic meter at a predetermined molecule measure. A characterization of the A to D grades can be found in below inserted table 1. The process chamber can thereby be classified according to a number and size of particles permitted per volume of gas. The number and size of particles can be measured by particle counting either based upon light scattering, light obscuration, or direct imaging. A high intensity light source is used to illuminate the particle as it passes through a detection chamber. The particle passes through the light source (typically a laser or halogen light) and if light scattering is used, then the redirected light is detected by a photo detector. If direct imaging is used, a halogen light illuminates particles from the back within a cell while a high definition, high magnification camera records passing particles. The recorded video is then analysed by computer software to measure particle attributes. If light blocking (obscuration) is used, a loss of light is detected. The amplitude of the light scattered or light blocked is measured and the particle is counted and tabulated into standardized counting bins. Direct imaging particle counting employs a use of a high resolution camera and a light to detect particles. Vision based particle sizing units obtain two-dimensional images that are analysed by computer software to obtain particle size measurement.

TABLE 1
EU GMPs Annex 1 Recommended Limits
for Particulate Contamination

| | Maximum permitted number of particles per m³ | | | |
|---|---|---|---|---|
| | equal to or greater than the tabulated size | | | |
| | At rest | | In operation | |
| Grade | 0.5 μm | 5.0 μm | 0.5 μm | 5.0 μm |
| A | 3,520 | 20 | 3.520 | 20 |
| B | 3,520 | 29 | 352,000 | 2,900 |
| C | 352,000 | 2,900 | 3,520,000 | 29,000 |
| D | 3,520,000 | 29,000 | Not defined | Not defined |

A comparison of the A to D grade room according to EU guidelines for good manufacturing practice for medicinal products, annex 1, and the FED-STD-209e (1992) Federal Standard 209-e Airborned Particulate Cleanliness Classes in Cleanrooms and Clean Zones, the ISO (1999) International Standard 14644-1: Cleanrooms and associated controlled environments-Part 1: Certification of Air Cleanliness. International Organisation for Standardisation, Switzerland, as well as the ISO (2003) International Standard ISO 14698-2:2003 Cleanrooms and associated controlled environments—Biocontamination control —Part 2: Evaluation and interpretation of biocontamination data is shown in below inserted Table 2.

TABLE 2
Comparison of classification systems
WHO Technical Report Series, No. 902,2002 Annex 6
Comparison of different airborne particulate
classification systems for clean areas^{a}

| WHO | United States | United States | ISO/TC | EEC |
|---|---|---|---|---|
| (GMP) | (209E) | (customary) | (209) | (GMP) |
| Grade A | M 3.5 | Class 100 | ISO 5 | Grada A |
| Grade B | M 3.5 | Class 100 | ISO 5 | Grade B |
| Grade C | M 5.5 | Class 10000 | ISO 7 | Grade C |
| Grade D | M 6.5 | Class 100000 | ISO 8 | Grade D |

EEC: European Commission; ISO/TC: International Organization for Standardization Technical Committee.
This comparison is defined based on at-rest limitations.

In an embodiment, the gas shower is laminar, which can be understood as essentially similar to a laminar flow or essentially unidirectional or as essentially not turbulent. The laminar gas shower or gas flow may be directed downwardly or in horizontal direction, preferably in a constant stream, towards a bottom of the process chamber. The laminar gas shower may allow reducing and maintaining extremely low levels of particulates, such as dust, airborne organisms, or vaporized particles, without introducing turbulences and/or contaminating e.g. a manufacturing procedure in the respective housing or chamber. Apparatus mounted in the area of the gas shower may be mounted tightly to the separation element or a process chamber wall or be covered or encapsulated to reduce or eliminate a turbulence or disturbance of the gas shower. Apparatus may also be “hidden” in an appendix of the separation element or in in an appendix of a process chamber wall to reduce or eliminate a turbulence or disturbance of the gas shower.

In an embodiment, the speed of the gas shower is at least 0.2 m/s. In an embodiment, the speed of the gas shower is in a range of about 0.2 to about 0.6 m/s, preferably about 0.36 to about 0.54 m/s. This speed may provide a good balance between reducing and maintaining extremely low levels of particulates and not disturbing e.g. a manufacturing procedure in the process chamber.

In an embodiment, the manufacturing device for a pharmaceutical product further comprises a first filter unit arranged upstream of the process chamber. “Upstream of the process chamber” can be understood as in front, before, or at an entrance of the process chamber. In the same or another embodiment, the manufacturing device comprises a second filter unit arranged between the process chamber and the technical chamber. There can be more filter units in these or other gas passages. The filter unit may be at least a filter and in particular, a high-efficiency particulate air (HEPA) filter. The filter unit may allow further reducing and maintaining extremely low levels of particulates, such as dust, airborne organisms, or vaporized particles in the gas flow. The filter unit may further prevent or reduce a backflow of particulates into cleaner compartments, e.g. back from the technical chamber into the process chamber.

The European Standard High efficiency airfilters (EPA, HEPA and ULPA)—Part 1: Classification, performance testing, marking; German version EN 1822-1:2009 defines several classes of HEPA filters by their retention at the given most penetrating particle size. A characterization of the filter classes can be found in below inserted table 3. The first filter unit may be at least a H14 filter based on the HEPA filter classes. The second filter unit may be at least a H13 filter based on the HEPA filter classes.

TABLE 3
Characterization of HEPA filter classes

| HEPA class | Retention (averaged) | Retention (spot) |
|---|---|---|
| H13 | >99.95% | >99.75% |
| H14 | >99.995% | >99.975% |

The filter unit may also be a cascade of filters, preferably arranged along the gas stream from a coarse to a fine filter. The filters can be HEPA filters or a HEPA filter can be used in conjunction with a pre-filter (not HEPA, e.g. carbon-activated) to extend the usage life of the more expensive HEPA filter. In such setup, the first stage in the filtration process may be a pre-filter, which removes larger particles from the gas flow. The second stage in the filtration process may be the high-quality HEPA filter, which removes finer particles that escape from the pre-filter.

In an embodiment, the control unit is configured to control the gas flow to provide in the process chamber at least 10 exchange volumes of gas per hour. In an embodiment, the control unit is configured to control the gas flow to provide in the process chamber about 20 to about 120 exchange volumes of gas per hour, preferably about 50 to about 100 exchange volumes of gas per hour. These exchange volumes may provide a good balance between reducing and maintaining extremely low levels of particulates in the process chamber and not disturbing e.g. a manufacturing procedure in the process chamber. The term “exchange volume of gas per hour” can be understood as how many volumes or contents of the process chamber are exchanged per hour. For example, an exchange volume per hour of 25 would lead for an exemplary process chamber volume of 1.5 m³ to an exchanged amount or volume of gas per hour of 37.5 m³/h.

In an embodiment, the control unit is configured to control the gas flow to provide in the process chamber a fresh gas supply of at least about 20 m³/h per m³ process chamber. Preferably, the control unit is configured to control the gas flow to provide in the process chamber a fresh gas supply of about 50 to about 200 m³/h, more preferably about 75 to about 150 m³/h per m³ process chamber. These rates of fresh gas supply may provide an improved balance between reducing and maintaining extremely low levels of particulates in the process chamber and not disturbing e.g. a manufacturing procedure in the process chamber.

In an embodiment, the control unit is configured to control the gas flow to provide a gas flow from the process chamber into the technical chamber. This can be understood in that the gas flows from the process chamber to and into the technical chamber. In the same or another embodiment, both chambers are arranged so that the gas flow in the technical chamber is parallel to the gas flow in the process chamber, but in an opposite direction. Preferably, there is the following gas flow within the housing: to the process chamber, into the process chamber, through the process chamber, out of the process chamber, to the technical chamber, into the technical chamber, through the technical chamber, and out of the technical chamber, while not all steps are necessary. The gas typically enters the housing before entering the process chamber. The gas then typically exits the housing after the technical chamber. Therefore, the manufacturing device may further comprise at least a duct through the housing for a passage of the gas flow between the housing and the environment, wherein the outlet duct may also be referred to as “exhaust”.

The duct may be an inlet and/or an outlet for the gas. Preferably, the duct is sealed relative to the environment. As stated above, the term “sealed” can be understood as dust-tight and protected against splashing of water as well as compliant with at least IP64. The seal is preferably a silicone seal. The inlet and the outlet ducts are preferably directly integrated in the housing of the manufacturing device. In case of coupling individual manufacturing devices, there is preferably not an inlet and an outlet duct for each manufacturing device but preferably only one inlet duct in the very first manufacturing device and an outlet duct in the very last manufacturing device, wherein the gas flows from one manufacturing device to the next.

At least a pump or a fan (or even a HVAC system) may be present at the inlet duct in order to allow for controlled (or, in other words, for actively controlled) ventilation of the gas (preferably the air) that enters the device. In addition, at least a pump or a fan (or even a HVAC system) may be present at the outlet duct in order to allow for controlled (or, in other words, for actively controlled) ventilation of the gas (preferably the air) that leaves the device. Considering this option of active control of ventilation at the inlet and/or outlet duct (preferably at both ducts), it is possible in a preferred embodiment to control the pressures inside the manufacturing device.

When it comes to the inlet and/or outlet duct, it is—in particular for the outlet duct—preferred that the duct comprises a filter system (such as e.g. a carbon filter) to ensure that no organic fumes or gases are leaving the device into the environment. Corresponding sensors might also be comprised in or at the duct in order to monitor the gas (preferably air) coming into and leaving the manufacturing device. When present, the environment as well as the personnel is protected from such potentially harmful organic fumes or gases.

The process chamber is separated from the technical chamber by the separation element. The process chamber may be suitable and used for a manufacturing of a pharmaceutical product. The technical chamber may be suitable and used for housing apparatus, as e.g. a pump, a motor, a mixer, a processor or the like. The separation element may be plate shaped. The separation element may be used to mount the apparatus thereto. Mounted apparatus may be covered to reduce a turbulence or disturbance of the gas flow, prevent precipitation and/or ease a cleaning. The separation element may extend (only) between the process chamber and the technical chamber or it may extend at least partially or completely through the housing. The separation element may also extend outside the housing to separate, in form of a septum, parts of the surrounding environment, as e.g. a surrounding cleanroom.

In an embodiment, the separation element comprises an appendix with an opening towards the process chamber and an extension in the direction of the technical chamber. The appendix may be used to house apparatus. This allows increasing the available space in the process chamber without increasing a length of the process chamber. Further, the gas flow may be not or less disturbed and/or precipitation surfaces are reduced. The appendix may also have an opening towards the technical chamber and an extension in the direction of the process chamber. In all cases, the appendix may be closable by a cover.

In an embodiment, the housing comprises a passage for the gas flow from the process chamber to the technical chamber. The passage can be understood as a channel for the gas.

The passage or channel may be provided as a duct or as an intermediate chamber arranged outside the process chamber and the technical chamber and (downstream) between the process chamber and the technical chamber. In a preferred embodiment, the passage or channel may be provided as a duct or as an intermediate chamber arranged outside the process chamber but inside the technical chamber such that in this embodiment the intermediate chamber is in fact not a separate chamber but part of the technical chamber. The intermediate chamber preferably comprises a filter element comprising, for instance an active carbon filter, to filter out a toxic composition such as e.g. organic solvents coming from the process chamber (to avoid e.g. discharge of such a composition in the technical chamber). Accordingly, there is no exposure to such a composition in the technical chamber, given that e.g. a maintenance work is carried out in the technical chamber.

The passage or channel may also transvers the separation element as a sealed conduit or sealed through hole. As stated above, the term “sealed” can be understood as dust-tight and protected against splashing of water as well as compliant with at least IP64. The sealed conduit may have the benefit to maintain low levels of particulates. The sealed conduit may also comprise a filter to further reduce a level of particulates and/or a valve unit to control the gas flow. The valve unit may be a valve, preferably a throttle valve.

In an embodiment, the housing comprises a blocking unit to block a passage for a gas flow from the technical chamber to the process chamber. The blocking unit may be arranged (downstream) behind the technical chamber and/or between the technical chamber and the process chamber. The blocking unit may be a valve, preferably a one-way valve, and more preferably a check valve. Optionally, the valve comprises air flaps. Preferably, the passage for a gas flow can therefore close to block the passage and thus the flow from gas into the process chamber, e.g. if maintenance work needs to be carried out. In one embodiment, the valve is configured to control the pressure such that the desired pressure can be adjusted by the control unit that also receives and provides signals to the valve (preferably in the form of a feedback loop).

In an embodiment, the control unit is configured to control the gas flow to provide in the technical chamber a gas exhaust of at least about 20 m³/h per m³ technical chamber. Preferably, the control unit is configured to control the gas flow to provide a gas exhaust out of the technical chamber of about 50 to about 200 m³/h, more preferably about 100 to about 300 m³/h, even more preferably about 200 to about 500 m³/h, most preferably about 300 to about 600 m³/h per m³ technical chamber. These exhaust rates may allow reducing and maintaining extremely low levels of particulates in the process chamber and increasing the manufacturing speed.

In an embodiment, the control unit is configured to control the gas flow to provide a pressure difference between the process chamber and the environment in a range of about 5 to about 100 Pa, preferably about 10 to about 20 or to about 15 Pa. The pressure difference may preferably be at least about 15 Pa. This can be understood in that there is a higher pressure in the process chamber than in the environment, including the technical chamber. At least the pressure in the process chamber can be higher than atmosphere pressure. The higher pressure in the process chamber makes it almost impossible for any particles to enter and contaminate the process chamber.

Further, the lower pressure in the technical chamber supports the aspects described below. In an embodiment, the control unit is configured to control the gas flow to provide a pressure difference between the technical chamber and the environment in a range of about −5 to about −100 Pa, preferably about −10 to about −20 Pa. The pressure difference may preferably be at least about −15 Pa. In an embodiment, the control unit is configured to control the gas flow to provide a pressure difference between the process chamber and the technical chamber in a range of about 10 to about 200 Pa, preferably about 20 to about 40 Pa. The pressure difference may preferably be at least about 30 Pa.

The above mentioned ranges of about 5 to about 100 Pa, preferably about 10 to about 20 Pa, more particularly at least about 15 Pa, as pressure difference between the process chamber and the environment are in particular suitable to support the following aspects:

In an embodiment, the technical chamber is dimensioned to provide a larger available space to the gas compared to the process chamber to expand the gas in the technical chamber to provide a lower pressure compared to the process chamber. In other words, the technical chamber is larger than the process chamber. Thereby, the pressure of the gas in the process chamber is automatically reduced when the gas flow leaves the process chamber and enters the technical chamber. As a result, the higher pressure in the process chamber makes it almost impossible for any particles from the technical chamber to enter and contaminate the process chamber. Further, the lower pressure in the technical chamber may form a suitable environment for the apparatus arranged in the technical chamber. Additionally, the gas flow in the technical chamber can be used to cool the technical chamber and in particular its apparatus, as e.g. a motor, a pump, a mixer, a processor etc.

In an embodiment, the technical chamber is dimensioned to provide a larger available space to the gas compared to the process chamber to suck the gas from the process chamber to the technical chamber. This means the larger available space for the gas in the technical chamber compared to the process chamber leads to an automatic streaming of the gas into the technical chamber. Preferably, no drive unit or pump is needed and thereby space and energy is saved.

In an embodiment, the technical chamber is dimensioned relative to the process chamber to expand the gas flow from the process chamber to cool the gas flow in the technical chamber relative to the process chamber due to the gas expansion. This means the expansion of the gas leads to a decrease of temperature. This may be in particular beneficial to cool the apparatus arranged in the technical chamber, as e.g. a processor, a motor, a mixer, a pump or the like.

In an embodiment, there is a higher pressure in the process chamber than in the technical chamber. This is fulfilled if either the pressure in the process chamber and the pressure in the technical chamber are higher than atmosphere pressure or the pressure in the process chamber is higher than atmosphere pressure and the pressure in the technical chamber is the same as atmosphere pressure or the pressure in the process chamber is higher than atmosphere pressure and the pressure in the technical chamber is below atmosphere pressure. In other words, in an embodiment, the control unit is configured to control the gas flow to provide in the technical chamber a negative pressure relative to the environment. This may further improve or strengthen the described aspects to expand the gas and to automatically suck the gas from the process chamber to the technical chamber.

In an embodiment, the manufacturing device for a pharmaceutical product further comprises a collect tray arranged at the passage from the process chamber to the technical chamber to collect liquid droplets. This is beneficial as humidity can be collected before harming the product or the apparatus arranged in the chamber. Humidity can be intentionally implemented or can derive from the gas expansion in the technical chamber. In an embodiment, the manufacturing device for a pharmaceutical product further comprises a humidity unit arranged at the passage from the process chamber to the technical chamber to control a humidity of the gas flow. The humidity unit may allow further controlling the humidity in the technical chamber to protect the apparatus in the technical chamber, as e.g. a processor, a motor, a mixer, a pump or the like.

In an embodiment, the process chamber comprises at least one waste channel, which includes a pump and a collection system for waste from the production and/or purification processes. Such a channel may furthermore comprise a sensor, such as e.g. a toxicity sensor, to analyze the waste in particular for organic waste that might be toxic. Such a channel might also be configured such that the separation between organic and non-organic waste is carried out.

In an embodiment, the manufacturing device for a pharmaceutical product further comprises a cooling unit configured to cool the gas flow in the technical chamber relative to the environment and/or the process chamber.

The cooling unit may allow having higher temperatures in the process chamber, e.g. for an improved manufacturing of medical applications, and/or lower temperatures in the technical chamber to protect the apparatus arranged in the technical chamber, as e.g. a processor, a motor, a pump, a mixer, or the like. The cooling unit and/or the following heating unit can be arranged within or outside the housing. In an embodiment, the manufacturing device for a pharmaceutical product further comprises a heating unit configured to heat the gas flow in the process chamber relative to the environment. The heating unit may allow higher temperatures in the process chamber to e.g. improve the manufacturing conditions for the medical applications.

In an embodiment, the manufacturing device for a pharmaceutical product further comprises a sensor unit comprising at least one of a group of a flow sensor, a pressure sensor, a temperature sensor, a humidity sensor and a leakage sensor. The sensor unit may be arranged in the housing, e.g. at the separation element, in the process chamber, in the technical chamber, in the intermediate chambers there between or the intermediate chamber as part of the technical chamber, in the conduits trough the separation element etc. The sensor unit may allow controlling the conditions in view of e.g. flow rate, pressure, temperature, humidity, leakage etc. Accordingly, the sensor unit may comprise at least one of a group of a flow sensor, a pressure sensor, a temperature sensor, a humidity sensor, a microbial sensor, a particulate sensor, an organics sensor, and a leakage sensor.

In an embodiment, the housing comprises at least a control cabinet arranged outside the process chamber and/or the technical chamber. The control cabinet may be openable by e.g. a swing door or a gullwing door. The control cabinet may comprise a control component for the process chamber and/or the technical chamber. This allows controlling and handling the procedure in the process chamber and/or the technical chamber without opening and disturbing the process chamber and/or the technical chamber.

In an embodiment, the housing comprises a door unit in the process chamber and/or in the technical chamber. The door unit may be openable to allow access into the respective chamber. The door unit may comprise a door and a door frame. The door may be a swing door to reduce the footprint of the manufacturing device. The door may be sealed relative to the door frame in a closed condition of the door to keep the chamber(s) clean and the manufacturing procedure safe. The seal may be a silicone frame. As stated above, the term “sealed” can be understood as dust-tight and protected against splashing of water as well as compliant with at least IP64. The door unit may further comprise a curtain, e.g. a (PVC) curtain arranged between the door and the gas shower to ensure an undisturbed seamless gas shower.

The door unit may comprise a door lock and in particular an electromagnetic lock to close and lock the door. In an embodiment, the door unit comprises a door hinge arranged between the door and the door frame. The door lock and/or the door hinge is preferably at least partially embedded into the door and/or the door frame to reduce surfaces which are detrimental to cleaning procedures. The door lock can in particular comprise a security seal such as e.g. an electromagnetic seal, which is closed during operating the manufacturing device to render opening of the door impossible when the production is ongoing. If the manufacturing device is not in operation, the door lock may be unlocked to allow opening the door unit.

In an embodiment, the door unit comprises a window directed into the process chamber and/or in the technical chamber. The window allows a non-invasive optical control of the procedures within the chamber(s), which is important for a stable and safe manufacturing procedure and good product quality. The door unit of the technical chamber (or the process chamber) may comprise a shutter configured to be closed in an open condition of the door and to be open in a closed condition of the door to guarantee a continuous gas flow in the other parts of the housing even in the open condition of the door.

In an embodiment, the manufacturing device for a pharmaceutical product further comprises an additive unit arranged within the housing and configured to add an additive to the gas flow. The additive unit may be arranged at the beginning or in the process chamber or the technical chamber. The additive unit may provide ozone or a cleaning additive, etc.

In an embodiment, the manufacturing device for a pharmaceutical product further comprises an attachment element arranged at an outer wall of the housing outside the housing. The attachment element may be configured to hold a media supply e.g. in form of a media reservoir. The manufacturing device may further comprise a piping for the media from the media reservoir to the interior of the housing, e.g. into the process chamber. The piping may be sealed relative to the housing wall. As stated above, the term “sealed” can be understood as dust-tight and protected against splashing of water as well as compliant with at least IP64. The piping may extend with a non-parallel angle (e.g. 3° to 5°) relative to the ground to allow a draining by gravitational force. In an embodiment, the manufacturing device further comprises a mounting element arranged at walls within the housing and in particular within the chambers. The mounting element may be configured to hold a media supply e.g. in form of a media container.

In an embodiment, the manufacturing device for a pharmaceutical product further comprises a coupling unit arranged at an outer wall of the housing and configured to couple the manufacturing device to another manufacturing device. The other manufacturing device may be similar to the above described manufacturing device for a pharmaceutical product or it might be a different device. The coupling unit may allow a mechanical coupling of at least two devices. The coupling unit may allow a fluid communication and coupling of at least two devices.

The coupling unit may allow a data communication and coupling of at least two devices. The fluid communication includes in particular the transport of a pharmaceutical product from one device to another device, for example the transport of a DNA template after its purification and/or filtration in a first device from this first device to a second device, wherein the DNA template may then be used in an RNA generation unit comprised in the second device.

Another example is the transport of RNA after its purification and/or filtration in a first device from this first device to a second device, wherein the RNA may then be used in a mixing unit resulting in formulated RNA comprised in the second device.

It is preferred that the coupling unit is also arranged to comprise a quality control unit, wherein this quality control unit is suitable for analyzing samples that are taken and/or provided from the communication, preferably the fluid communication between the devices. Thus, as exemplified above, if e.g. a DNA template is transported from a first device to a second device, a sample may be taken and analyzed by the quality control unit, which is preferably located in or at the coupling unit.

### Production of any Pharmaceutical Product

In general, the manufacturing device for a pharmaceutical product is suitable to produce any pharmaceutical product. Thus, the manufacturing device of the present invention provides the conditions that are required to produce any pharmaceutical product, in particular by providing a process chamber (corresponding to the wet part, where inter alia the reactions and purifications take place) with—when the device is in use—a positive pressure relative to the environment and a gas shower falling in the direction of gravity such that the contamination is kept to a minimum where the production takes place. Furthermore, a technical chamber is provided (corresponding to the dry part, where inter ala the technical media is located), which—when the device is in use—provides the technical media supply for the units and apparatus in the process chamber, optionally via connections through sealed holes in the separation element.

The manufacturing device is rather small such that it can be shipped and thus installed and operated in a flexible manner as outlined above, and it can be equipped with any units and any apparatus to produce a desired pharmaceutical product. The skilled person will be able to select such units and apparatus depending on the product.

### Production of an RNA-Based Pharmaceutical Product

It may be desirable to produce an RNA-based pharmaceutical product, such as in particular an RNA vaccine (e.g. a lipid nanoparticle encapsulated RNA vaccine). In essence, the production of the RNA vaccine can be initiated once the sequence of the target is known, e.g. the sequence of a viral protein (e.g. the spike protein in case of SARS-CoV₂). The process then typically follows the steps of i) de-novo synthesis of the DNA with the desired sequence; ii) DNA template generation; iii) RNA generation; iv) encapsulation of the RNA into the formulation; and v) filling and finishing the product, wherein each step builds upon the previous step. Of course, typical purification and filtration step will also be carried out in between the afore-mentioned steps, if applicable.

In this embodiment, the manufacturing device for a pharmaceutical product further comprises at least one of a group comprising a process media supply unit, a mixing unit, a de-novo DNA synthesis unit, a DNA template generation unit, an RNA generation unit, a formulation unit, a purification unit, a filtration unit, a fill-and-finish unit, and combinations thereof.

The following embodiments relate to the units disclosed herein.

In a preferred embodiment, the process media supply unit comprises a mounting element configured to hold a process medium supply container in the process chamber. Typically, there are several mounting elements, each configured to hold a process medium supply container, which is then connected, e.g. via sterile tubes, to the downstream unit or device, such as e.g. the DNA de-novo synthesis unit, the DNA template generation unit, the RNA generation unit or the formulation unit. The downstream unit or device may also be a purification unit, such as e.g. a reversed-phase HPLC column. The process medium can be selected from the group consisting of synthetic DNA for amplification in a PCR reaction, a PCR component mix, water, in-vitro-translation buffer, a nucleotide including UTP, GTP, ATP and CTP, an enzyme including an RNA polymerase, a buffer including a wash buffer, a reagent buffer (e.g. an immobilization buffer) and an elution buffer, HPLC buffers, a formulation solution including a lipid solution, and an HPLC eluent.

It is important to understand that the technical media supply differs from the process media supply in that the technical media supply is located in the technical chamber and has no direct contact with any process medium or reactant(s) and/or product(s) thereof. The technical medium can be selected from power cables, pumps and cooling fans, sanitizing agents, gas, and the like.

In a preferred embodiment, the mixing unit comprises a mixer. It may also comprise a pump unit (e.g. a syringe pump, pulseless flow pump, peristaltic pump). The mixing unit may e.g. be used in combination with either the DNA template generation unit or the RNA generation unit, where the mixing unit is present upstream of either the DNA template generation unit or the RNA generation unit and provides the respective master mixes for the subsequent DNA template generation unit or the RNA generation unit. A mixing unit may also be used when a polyvalent pharmaceutical product is produced, such as e.g. a polyvalent RNA-based pharmaceutical product that comprises at least two different RNA sequences. In the case of such polyvalent RNA-based pharmaceutical products, a mixing unit may be used to mix the at least two different RNAs (i.e. the at least two RNAs with different sequences) prior to these RNAs being formulated in the formulation unit. Alternatively, at least two different RNAs that are already formulated may be mixed by a mixing unit in order to obtain a polyvalent RNA-based pharmaceutical product.

The mixing unit may also be used when formulating the nucleic acid product, in particular the RNA, and may in this case be referred to as formulation unit. Thus, the mixing unit may be used for formulation when lipid nanoparticle (LNP) or liposome encapsulated RNA or RNA complexed with a polycationic peptide or protein (e.g. protamine or a polymeric carrier, e.g. a polyethylene glycol/peptide polymer e.g. according to WO2012/013326) is generated. In the context of formulation, the mixing unit may comprise at least two pump units (e.g. one unit for pumping the lipids, one unit for pumping the RNA), and, optionally, a reactor for complexation/formulation (e.g. a T-piece connector).

In a preferred embodiment, the de-novo DNA synthesis unit comprises a solid-phase synthesis unit for DNA, typically using the phosphoramidite method and corresponding phosphoramidite building blocks derived from protected 2′-deoxynucleosides in order to obtain DNA-oligonucleotides of about 200 nucleotides in length.

Preferably in a fully automated manner, the phosphoramidite building blocks are sequentially coupled according to the sequence that is to be produced (which may also be referred to as in silico designed sequence), wherein the oligonucleotide-product is typically released from the solid phase to solution, then deprotected and collected. The de-novo DNA synthesis unit may furthermore be configured to assemble (or couple) at least two oligonucleotides obtained by the phosphoramidite method in order to obtain longer sequences, if applicable. As noted above, a purification unit will typically follow the de-novo DNA synthesis unit. De-novo DNA synthesis may also be based on enzymatic processes e.g. based on terminal deoxynucleotidyl transferase (TdT) as described in WO2015159023. Additionally, the de-novo DNA synthesis unit may comprise a ligation unit for ligating the de-novo synthetized DNA into an appropriate DNA backbone.

In a preferred embodiment, the DNA template generation unit comprises a polymerase chain reaction unit. In an embodiment, the DNA template generation unit is configured to generate sufficient amount of DNA template suitable for use in the RNA generation unit. In a preferred embodiment, the DNA template generation unit may comprise a thermocycler element for PCR-based DNA amplification. Suitably, said DNA template generation unit may generate DNA templates, preferably PCR-based DNA templates. In an embodiment, the DNA template is an end-modified or end-functionalised PCR-generated DNA template. Preferably, the DNA template is a biotinylated PCR-generated DNA template, a non-modified or end-modified linearized plasmid DNA or a non-modified or end-modified linearized doggy bone DNA.

In a preferred embodiment, the RNA generation unit comprises a bioreactor for RNA in vitro transcription, preferably a bioreactor as disclosed in WO 2020/002598. In particular, a bioreactor as defined by Claims 1 to 59 in WO 2020/002598, or as illustrated by FIGS. 1 to 14 in WO 2020/002598 may be used as an RNA generation unit. It is especially preferred for the bioreactor that the DNA used as template in the bioreactor is immobilized on magnetic particles and that the bioreactor comprises a magnet for mixing the reaction solution including the DNA immobilized on magnetic particles.

In a preferred embodiment, the purification unit comprises an HPLC unit, preferably a unit for performing RP-HPLC. Particularly preferred in that context is RP-HPLC using a method disclosed in WO2008/077592 preferably using a porous, non-alkylated poly(stryrene-divinylbenzene) reverse phase, wherein the reverse phase is formed by beads or occurs as a polymerized block (e.g. monolithic). Alternatively, or in addition, the purification unit may comprise an affinity chromatography unit, preferably an oligo dT purification unit for affinity purification of polyadenylated nucleic acid, in particular RNA, via oligo dT functionalized matrices or beads or columns (e.g. as described in WO2014152031A1). Alternatively, or in addition, the purification unit may comprise an anion exchange chromatography unit. Alternatively, or in addition, the purification unit may comprise a unit for nucleic acid precipitation and a unit for purifying said precipitated nucleic acid (e.g. using TFF or centrifugation or filtration).

Alternatively, or in addition, the purification unit may comprise a unit for dsRNA removal e.g. a unit that involves RNase III treatment or a unit that involves a step of cellulose-based dsRNA purification. The purification unit may be used to purify in particular a nucleic acid, preferably the DNA obtained in the de-novo DNA synthesis unit, the template DNA obtained in the DNA template generation unit and/or the RNA obtained in the RNA generation unit.

In preferred embodiments, the purification unit, in particular the purification unit for purifying RNA, comprises an RP-HPLC unit and an oligo dT purification unit and, optionally, a unit for dsRNA removal.

In a preferred embodiment, the filtration unit comprises a tangential-flow filtration unit. Particularly preferred in that context is tangential flow filtration as described in WO2016/193206, wherein TFF is used for diafiltration and/or concentration and/or purification of a nucleic acid. The filtration unit may be used to filter in particular a nucleic acid, preferably the template DNA or the RNA, after the purification unit. It can also be preferred that the filtration unit comprises a sterile filter, preferably a sterile filter with a size of 0.22 μm, in order to sterile-filter in particular a formulation comprising the RNA, in particular mRNA, more particularly LNP-formulated mRNA.

In a preferred embodiment, the fill-and-finish unit comprises a filling and/or dosing machine. The fill-and-finish unit is configured to fill a formulated pharmaceutical product, in particular formulated active pharmaceutical ingredient (in particular the formulated RNA, more particularly the LNP-formulated mRNA) into suitable vials, such as glass vials in an amount that corresponds to a single dose or multiple doses of the formulated active pharmaceutical ingredient. Typically the vials, such as the glass vials, are closed with caps (e.g. aluminium caps) under sterile conditions. In embodiments, the fill-and-finish unit comprises a freezing unit for freezing the obtained drug product to at least −20° C., preferably to at least −60° C. or −80° C.

In an embodiment, the manufacturing device for a pharmaceutical product further comprises a process media supply unit, a mixing unit, a de-novo DNA synthesis unit, a purification unit and a filtration unit, wherein the units are preferably connected in the order as listed and the device is preferably configured to produce DNA. In an embodiment, the manufacturing device for a pharmaceutical product further comprises a process media supply unit, a mixing unit, a DNA template generation unit, a purification unit and a filtration unit, wherein the units are preferably connected in the order as listed and the device is preferably configured to produce template DNA.

In an embodiment, the manufacturing device for a pharmaceutical product further comprises a process media supply unit, a mixing unit, an RNA generation unit, a purification unit and a filtration unit, wherein the units are preferably connected in the order as listed and the device is preferably configured to produce RNA, preferably mRNA.

In an embodiment, the manufacturing device for a pharmaceutical product further comprises a process media supply unit, a formulation unit, a purification unit and a filtration unit, wherein the units are preferably arranged in the order as listed and the device is preferably configured to produce formulated RNA, preferably formulated mRNA, more preferably LNP-formulated mRNA.

In an embodiment, the manufacturing device for a pharmaceutical product further comprises a process media supply unit, a mixing unit, a de-novo DNA synthesis unit, a DNA template generation unit, a purification unit, a filtration unit, a formulation unit and an RNA generation unit, wherein the units are preferably arranged in the order of i) a process media supply unit, ii) a mixing unit, iii) a de-novo DNA synthesis unit, iv) a purification unit, v) a filtration unit, vi) a process media supply unit, vii) a mixing unit, viii) a DNA template generation unit, ix) a purification unit, x) a filtration unit, xi) a process media supply unit, xii) a mixing unit, xiii) an RNA generation unit, xiv) a purification unit, xv) a filtration unit, xvi) a process media supply unit, xvii) a formulation unit, xviii) a purification unit and xix) a filtration unit and the device is preferably configured to produce formulated RNA, preferably formulated mRNA, more preferably LNP-formulated mRNA.

In an especially preferred embodiment, each unit comprises a technical media supply, wherein the technical media supply is located in the technical chamber, a process media supply and/or a device configured to be in contact with the process media, wherein the process media supply and the device are located in the process chamber, and a sealed through hole located in the separation element and configured to connect the technical media supply and the device configured to be in contact with the process media. A unit may furthermore comprise a waste outlet, where e.g. wash buffer or by-products are collected.

In a further embodiment, the manufacturing device according to the present invention may comprise at least one of a DNA immobilization unit e.g. for immobilizing plasmid DNA (e.g. as described in WO2019122371), a DNA linearization unit e.g. for linearization of plasmid DNA or doggy bone DNA (e.g. by using restriction enzymes as described in WO2016174227), an RNA capping unit e.g. for adding a cap0 or cap1 structure to in vitro transcribed RNA (e.g. by using immobilized capping enzymes as described in WO2016193226), an RNA polyadenylation unit e.g. for adding a polyA tail to in vitro transcribed RNA (e.g. by using immobilized RNA polyadenylation enzymes as described in WO2016174271), a combined capping-tailing unit for adding a cap0 or cap1 structure and a polyA tail to in vitro transcribed RNA, an RNA mixing unit e.g. for mixing at least two different RNA species, an RNA spray drying unit e.g. for generating spray-dried or freeze-spray dried RNA (e.g. according to WO2016/184575 or WO2016184576), an RNA lyophilization unit for generating lyophilized RNA (e.g. according to WO2016/165831 or WO2011/069586), and/or a unit for end-product storage. In an embodiment, the manufacturing device according to the present invention further comprises at least one of an NGS (next generation sequencing) unit e.g. for sequence analysis, a mass-spectrometry (MS) unit, a quality control unit (e.g. comprising an HPLC unit for analytical HPLC), a qPCR or ddPCR unit, a capillary electrophoresis unit, a media supply rack or a media supply unit, a documentation unit and/or a unit for computer assisted control for all processing steps and interfaces for higher order controls and documentation systems.

In a second aspect, the present invention is directed to a manufacturing module for a pharmaceutical product.

The manufacturing module comprises at least two manufacturing devices as described above. The at least two manufacturing devices are coupled with each other. The coupling may allow a mechanical coupling of the at least two manufacturing devices. The coupling may allow a fluid communication and coupling of the at least two manufacturing devices. The coupling may allow a data communication and coupling of the at least two manufacturing devices. The fluid communication includes in particular the transport of a pharmaceutical product from one device to another device, for example the transport of a DNA template after its purification and/or filtration in a first device from this first device to a second device, wherein the DNA template may then be used in an RNA generation unit comprised in the second device. Another example is the transport of RNA after its purification and/or filtration in a first device from this first device to a second device, wherein the RNA may then be used in a mixing unit resulting in formulated RNA comprised in the second device. As noted above, it is preferred that quality control takes place at the coupling sites, e.g. to check the quality of the DNA template or of the RNA prior to these entering the next device, preferably by taking samples from the fluid communication between the devices.

In an embodiment, the manufacturing module for a pharmaceutical product further comprises at least one of a group comprising a process media supply unit, a mixing unit, a de-novo DNA synthesis unit, a DNA template generation unit, an RNA generation unit, a purification unit, a filtration unit, a fill-and-finish unit, and combinations thereof.

In an embodiment, the manufacturing module for a pharmaceutical product further comprises a process media supply unit, a mixing unit, a de-novo DNA synthesis unit, a purification unit and a filtration unit, wherein the units are preferably connected in the order as listed and the module is preferably configured to produce DNA.

In an embodiment, the manufacturing module for a pharmaceutical product further comprises a process media supply unit, a mixing unit, a DNA template generation unit, a purification unit and a filtration unit, wherein the units are preferably connected in the order as listed and the module is preferably configured to produce template DNA.

In an embodiment, the manufacturing module for a pharmaceutical product further comprises a process media supply unit, a mixing unit, an RNA generation unit, a purification unit and a filtration unit, wherein the units are preferably connected in the order as listed and the module is preferably configured to produce RNA, preferably mRNA.

In an embodiment, the manufacturing module for a pharmaceutical product further comprises a process media supply unit, a formulation unit, a purification unit and a filtration unit, wherein the units are preferably arranged in the order as listed and the module is preferably configured to produce formulated RNA, preferably formulated mRNA, more preferably LNP-formulated mRNA.

In an embodiment, the manufacturing module for a pharmaceutical product further comprises a process media supply unit, a mixing unit, a de-novo DNA synthesis unit, a DNA template generation unit, a purification unit, a filtration unit, a formulation unit and an RNA generation unit, wherein the units are preferably arranged in the order of i) a process media supply unit, ii) a mixing unit, iii) a de-novo DNA synthesis unit, iv) a purification unit, v) a filtration unit, vi) a process media supply unit, vii) a mixing unit, viii) a DNA template generation unit, ix) a purification unit, x) a filtration unit, xi) a process media supply unit, xii) a mixing unit, xiii) an RNA generation unit, xiv) a purification unit, xv) a filtration unit, xvi) a process media supply unit, xvii) a formulation unit, xviii) a purification unit and xix) a filtration unit and the module is preferably configured to produce formulated RNA, preferably formulated mRNA, more preferably LNP-formulated mRNA.

In an embodiment, the manufacturing module for a pharmaceutical product comprises a first manufacturing device comprising a process media supply unit and a mixing unit, a second manufacturing device comprising a de-novo DNA synthesis unit, a third manufacturing device comprising a purification unit and a fourth manufacturing device comprising a filtration unit. In an embodiment, the manufacturing module is further configured to produce DNA.

In an embodiment, the manufacturing module for a pharmaceutical product comprises a first manufacturing device comprising a process media supply unit and a mixing unit, a second manufacturing device comprising a DNA template generation unit, a third manufacturing device comprising a purification unit and a fourth manufacturing device comprising a filtration unit. In an embodiment, the manufacturing module is further configured to template DNA.

In an embodiment, the manufacturing module for a pharmaceutical product comprises a first manufacturing device comprising a process media supply unit and a mixing unit, a second manufacturing device comprising a bioreactor for RNA in vitro transcription, a third manufacturing device comprising a purification unit and a fourth manufacturing device comprising a filtration unit, the fourth device preferably comprising two filtration units, namely a tangential flow filtration unit and a sterile filter. In an embodiment, the manufacturing module is further configured to produce RNA, preferably mRNA.

In an embodiment, the manufacturing module for a pharmaceutical product comprises a first manufacturing device comprising a process media supply unit and a formulation unit, a second manufacturing device comprising a filtration unit, preferably a tangential flow filtration unit, and a third manufacturing device comprising a filtration unit, preferably a sterile filter. In an embodiment, the manufacturing module is further configured to produce formulated RNA, preferably formulated mRNA, more preferably LNP-formulated mRNA.

In an embodiment, the manufacturing module for a pharmaceutical product comprises a first manufacturing device comprising a process media supply unit and a mixing unit, a second manufacturing device comprising a DNA template generation unit, a third manufacturing device comprising a purification unit, a fourth manufacturing device comprising a filtration unit, a fifth manufacturing device comprising a process media supply unit and a mixing unit, a sixth manufacturing device comprising a bioreactor for RNA in vitro transcription, a seventh manufacturing device comprising a purification unit, an eight manufacturing device comprising a filtration unit, a ninth manufacturing device comprising a process media supply unit and a mixing unit, a tenth manufacturing device comprising a filtration unit, preferably a tangential flow filtration unit, and an eleventh manufacturing device comprising a filtration unit, preferably a sterile filter. When starting from the de-novo DNA synthesis, the manufacturing module further comprises as the very first devices i) a manufacturing device comprising a process media supply unit and a mixing unit, ii) a manufacturing device comprising a de-novo DNA synthesis unit, iii) a manufacturing device comprising a purification unit, and iv) a manufacturing device comprising a filtration unit, then followed by the first and further devices as set out in the previous embodiment. In an embodiment, the manufacturing module is further configured to produce formulated RNA, preferably formulated mRNA, more preferably LNP-formulated mRNA.

In a third aspect, the present invention is directed to a manufacturing system for a pharmaceutical product. The manufacturing system comprises a manufacturing device or a manufacturing module as described above as well as a clean room. The manufacturing device or the manufacturing module is arranged in the clean room.

In an embodiment, the clean room is configured to be at least a grade D room according to the above mentioned EU guidelines for good manufacturing practice for medicinal products, annex 1. Preferably, the clean room is configured to be a grade D or a grade C room.

In an embodiment, the clean room is a tent, a cell or a shippable container. This allows also the transporting of the entire manufacturing system to e.g. an emergency site. Further, the transport is safe and no time-consuming preparation, cleaning, adjustment and/or calibration of the manufacturing system is needed.

In a fourth aspect, the present invention is directed to methods of manufacturing.

Thus, the present invention includes a method for producing a pharmaceutical product, wherein the method comprises the following steps:
providing a manufacturing device according to the first aspect or any embodiment thereof or a manufacturing module according to the second aspect or any embodiment thereof or a manufacturing system according to the third aspect or any embodiment thereof; wherein the device, module or system comprises a unit for the production of the pharmaceutical product, wherein a technical media supply of the unit is located in a technical chamber, a process media supply of the unit and a device configured to be in contact with the process media are located in a process chamber, and a separation element comprises a sealed through hole configured to connect the technical media supply and the device of the unit;
providing a gas flow through the process chamber, wherein the gas flow through the process chamber is a gas shower falling in the direction of gravity;
providing a positive pressure in the process chamber relative to the environment;
providing a process media and a technical media for the unit; and
operating the unit to obtain the pharmaceutical product.

It is preferred in the above method that more than one unit is comprised in the device, module or system, wherein the units are chosen in their order and properties in accordance with the production steps of the pharmaceutical product.

The present invention also includes a method for producing DNA, wherein the method comprises the following steps:
providing a manufacturing device according to embodiments of the first aspect or a manufacturing module according to embodiments of the second aspect or a manufacturing system according to embodiments of the third aspect, wherein the embodiments relate to the de-novo synthesis of DNA,
providing a gas flow through a process chamber, wherein the gas flow through the process chamber is a gas shower falling in the direction of gravity;
providing a positive pressure in the process chamber relative to the environment;
providing a process media and a technical media for the units; and
mixing the process media required for the de-novo DNA synthesis in a mixing unit, followed by the de-novo synthesis of DNA in a de-novo DNA synthesis unit, followed by purifying the DNA in a purification unit and by filtering the DNA in a filtering unit to obtain the DNA.

The present invention also includes a method for producing a DNA template, wherein the method comprises the following steps:
providing a manufacturing device according to embodiments of the first aspect or a manufacturing module according to embodiments of the second aspect or a manufacturing system according to embodiments of the third aspect, wherein the embodiments relate to the production of a DNA template,
providing a gas flow through a process chamber, wherein the gas flow through the process chamber is a gas shower falling in the direction of gravity;
providing a positive pressure in the process chamber relative to the environment;
providing a process media and a technical media for the units; and
mixing the process media required for a PCR mastermix in a mixing unit, followed by generating a DNA template in a DNA template generation unit, followed by purifying the DNA template in a purification unit and by filtering the DNA template in a filtering unit to obtain the DNA template.

The present invention also includes a method for producing an RNA, wherein the method comprises the following steps:
providing a manufacturing device according to embodiments of the first aspect or a manufacturing module according to embodiments of the second aspect or a manufacturing system according to embodiments of the third aspect, wherein the embodiments relate to the production of an RNA,
providing a gas flow through a process chamber, wherein the gas flow through the process chamber is a gas shower falling in the direction of gravity;
providing a positive pressure in the process chamber relative to the environment;
providing a process media and a technical media for the units; and
mixing the process media required for an IVT mastermix in a mixing unit, followed by adding a template DNA and generating an RNA in an RNA generation unit, followed by purifying the RNA in a purification unit and by filtering the RNA in a filtering unit, optionally in two filtering units, namely a tangential flow filtration unit and a sterile filter, to obtain the RNA.

The present invention also includes a method for producing a formulated RNA, wherein the method comprises the following steps:
providing a manufacturing device according to embodiments of the first aspect or a manufacturing module according to embodiments of the second aspect or a manufacturing system according to embodiments of the third aspect, wherein the embodiments relate to the production of formulated RNA,
providing a gas flow through a process chamber, wherein the gas flow through the process chamber is a gas shower falling in the direction of gravity;
providing a positive pressure in the process chamber relative to the environment;
providing a process media and a technical media for the units; and
mixing the process media, preferably a lipid solution, and an RNA in a formulation unit, followed by filtering a formulated RNA in a tangential flow filtration unit and by filtering the formulated RNA in a sterile filter to obtain the formulated RNA.

The present invention also includes a method for producing a formulated RNA, wherein the method comprises the following steps:
providing a manufacturing device according to embodiments of the first aspect or a manufacturing module according to embodiments of the second aspect or a manufacturing system according to embodiments of the third aspect, wherein the embodiments relate to the production of formulated RNA,
providing a gas flow through a process chamber, wherein the gas flow through the process chamber is a gas shower falling in the direction of gravity;
providing a positive pressure in the process chamber relative to the environment;
providing a process media and a technical media for the units; and
mixing the process media required for the de-novo DNA synthesis in a mixing unit, followed by the de-novo synthesis of DNA in a de-novo DNA synthesis unit, followed by purifying the DNA in a purification unit and by filtering the DNA in a filtering unit, followed by mixing the process media required for a PCR mastermix in the mixing unit, followed by adding the de-novo synthesized DNA and by generating a DNA template in a DNA template generation unit, followed by purifying the DNA template in a purification unit and by filtering the DNA template in a filtering unit, followed by mixing the process media required for an IVT mastermix in the mixing unit, followed by adding the template DNA and generating an RNA in a RNA generation unit, followed by purifying the RNA in a purification unit and by filtering the RNA in a filtering unit, optionally in two filtering units, namely a tangential flow filtration unit and a sterile filter, followed by mixing the process media, preferably a lipid solution, and the RNA in the mixing unit, followed by filtering a formulated RNA in the tangential flow filtration unit and by filtering the formulated RNA in the sterile filter to obtain the formulated RNA.

In an embodiment, the above methods include a step of sanitizing the process chamber after obtaining the respective pharmaceutical product, wherein this step is preferably automated.

In an embodiment, the above methods are carried out under GMP-compliant conditions.

In an embodiment, the above methods include a step of analyzing the obtained respective pharmaceutical product for its quality, in particular by analytical methods to determine quality parameters such as e.g. the amount, integrity or purity. Such a step of analyzing may of course also apply to any intermediate products, in particular the synthesized DNA, the generated template DNA, the generated RNA, and so on.

In a fifth aspect, the present invention is directed to uses of the manufacturing device as described above or the manufacturing module as described above or the manufacturing system.

Thus, the present invention includes a use of a manufacturing device according to the first aspect or any embodiment thereof or a manufacturing module according to the second aspect or any embodiment thereof or a manufacturing system according to the third aspect or any embodiment thereof for the production of a pharmaceutical product. The pharmaceutical product may be formulated. The pharmaceutical product is preferably an active pharmaceutical ingredient in the form of a biomolecule or any precursor or any intermediate thereof, wherein the biomolecule is preferably a peptide, a protein or a nucleic acid, more preferably RNA, and most preferably mRNA. The production is preferably GMP-compliant.

The present invention also includes a use of a manufacturing device according to embodiments of the first aspect or a manufacturing module according to embodiments of the second aspect or a manufacturing system according to embodiments of the third aspect, wherein the embodiments relate to the production of a DNA template, for the production of DNA, preferably a DNA template. The production is preferably GMP-compliant

The present invention also includes a use of a manufacturing device according to embodiments of the first aspect or a manufacturing module according to embodiments of the second aspect or a manufacturing system according to embodiments of the third aspect, wherein the embodiments relate to the production of an RNA, preferably for the production of mRNA. The production is preferably GMP-compliant.

The present invention also includes a use of a manufacturing device according to embodiments of the first aspect or a manufacturing module according to embodiments of the second aspect or a manufacturing system according to embodiments of the third aspect, wherein the embodiments relate to the production of formulated RNA, preferably formulated mRNA, more preferably LNP-formulated mRNA. The production is preferably GMP-compliant.

In an embodiment, the above uses are automated. This means the use or the operation of the manufacturing device as described above or the manufacturing module as described above or the manufacturing system as described above can be fully or partially automated. This may allow a more reliable, faster and/or more cost effective manufacturing of a pharmaceutical product.

It should be noted that the aspects of the invention described above apply to the manufacturing device; the manufacturing module comprising at least two manufacturing devices; the manufacturing system comprising a manufacturing device or a manufacturing module and a clean room; the method for producing DNA, preferably template DNA; the method for producing RNA, preferably mRNA; the method for producing formulated RNA, preferably LNP-formulated RNA; the use of the manufacturing device, the manufacturing module or the manufacturing system for the production of a pharmaceutical product; the use of the manufacturing device, the manufacturing module or the manufacturing system for the production of DNA, preferably template DNA; the use of the manufacturing device, the manufacturing module or the manufacturing system for the production of RNA, preferably mRNA; and the use of the manufacturing device, the manufacturing module or the manufacturing system for the production of formulated RNA, preferably LNP-formulated RNA. It shall be understood that the device, the module, the system, the method, and the use according to the independent claims have similar and/or identical preferred embodiments, in particular, as defined in the dependent claims. It shall be understood further that a preferred embodiment of the invention can also be any combination of the dependent claims with the respective independent claim.

These and other aspects of the present invention will become apparent from and be elucidated with reference to the embodiments described hereinafter.

### BRIEF DESCRIPTION OF THE DRAWINGS

The Figures shown in the following are merely illustrative and shall describe the present invention in a further way.

These figures shall not be construed to limit the present invention thereto.

FIG. 1a shows schematically and exemplarily an embodiment of a manufacturing device for a pharmaceutical product according to the invention in a cross-section.

FIG. 1b shows schematically and exemplarily another embodiment of a manufacturing device for a pharmaceutical product according to the invention in a cross-section.

FIG. 2 shows schematically and exemplarily an embodiment of a manufacturing device for a pharmaceutical product according to the invention in a cross-section.

FIG. 3 shows schematically and exemplarily an embodiment of a manufacturing device for a pharmaceutical product according to the invention in a side view.

FIG. 4 shows schematically and exemplarily a detail of a manufacturing device for a pharmaceutical product according to the invention.

FIG. 5a shows a three dimensional view of the separation element.

FIG. 5b shows another three dimensional view of the separation element.

FIG. 6 shows schematically and exemplarily a manufacturing module for a pharmaceutical product according to the invention.

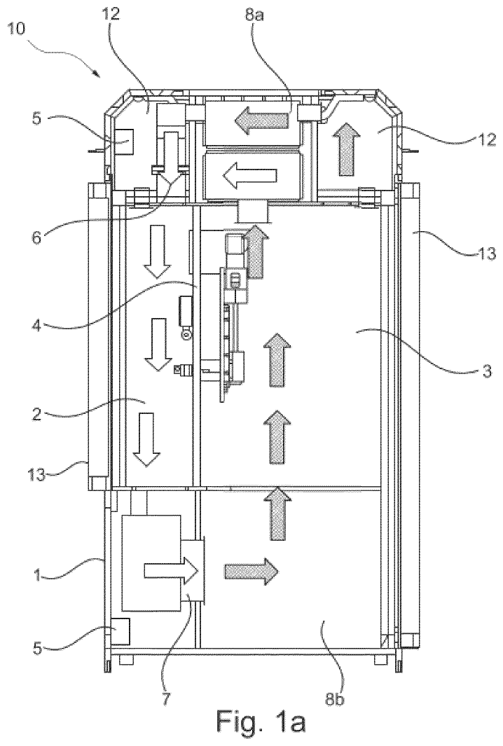

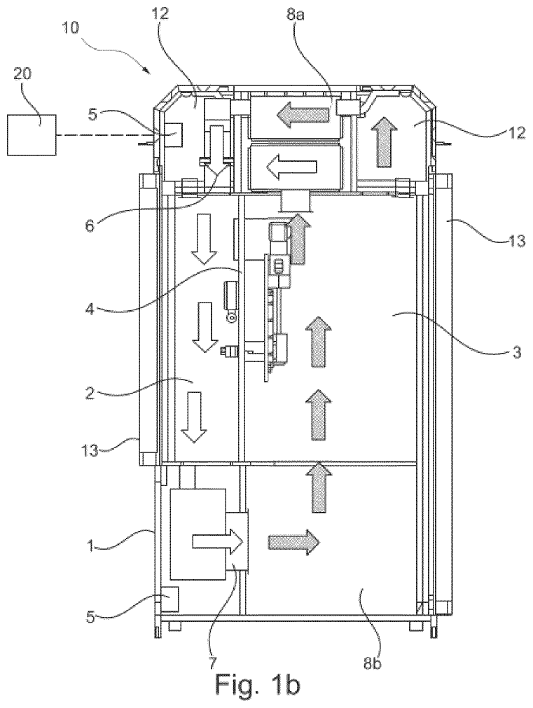

FIGS. 1a, 1b and 2 show cross-sections through a manufacturing device 10 for a pharmaceutical product according to the invention. The manufacturing device 10 comprises a housing 1, a process chamber 2, a technical chamber 3, a separation element 4, and a control unit 5. The pharmaceutical product may be an active pharmaceutical ingredient in form of a biomolecule or any precursor or any intermediate thereof. The manufacturing device 10 may be used for a vaccine production, in particular RNA vaccines and mRNA-based vaccines, from raw materials to a final product. A human operator may be positioned outside the manufacturing device to monitor the manufacturing process (FIG. 2).

The housing 1 is closed relative to the environment outside the housing 1. The housing 1 is sealed relative to the environment. As stated above, the term “sealed” can be understood as compliant with at least IP64. The housing 1 encompasses the process chamber 2 and the technical chamber 3.

The process chamber 2 may be suitable and used for manufacturing the pharmaceutical product. The process chamber 2 is here a grade A room. The process chamber 2 is sealed relative to the technical chamber 3. “Sealed” can be understood as compliant with at least IP64.

The technical chamber 3 may be suitable and used for housing apparatus, as e.g. a pump, a motor, a mixer, a processor or the like. The technical chamber 3 and the process chamber 2 are arranged so that the gas flow in the technical chamber 3 is parallel to the gas flow in the process chamber 2, but in an opposite direction (anti-parallel, see arrows in FIGS. 1 and 2).

The separation element 4 separates the process chamber 2 from the technical chamber 3. The separation element 4 has a plate-shape. The separation element 4 extends through the housing 1. The separation element 4 is used to mount apparatus of the process chamber 2 and the technical chamber 3 thereto.

The at least one, here two control units 5 control the gas flow through the process chamber 2, control the gas flow to provide a positive pressure in the process chamber 2 and control the gas flow to provide a gas shower through the process chamber 2. The control unit 5 can be a valve or a processor. The gas flow is provided by a flow unit 20. The flow unit 20 may be arranged within the housing (as in FIG. 1a, but not explicitly shown) or outside the housing and may communicate with at least one of the control units 5 (see FIG. 1b). The flow unit 20 may be e.g. a pump or a HVAC system. The flow unit 20 provides variable volumes of gas, while the control unit 5 adjusts different pressures in the gas flow. The control unit 5 controls the gas flow (arrows in FIGS. 1 and 2, white arrows for clean gas, grey arrows for less clean gas after leaving the process chamber 2) within the housing 1 as follows:
from an upper intermediate chamber 8a to the process chamber 2,
into the process chamber 2,
through the process chamber 2,
out of the process chamber 2,
into a lower intermediate chamber 8b,
out of the lower intermediate chamber 8b,
to the technical chamber 3,
into the technical chamber 3,
through the technical chamber 3,
out of the technical chamber 3, and
into the upper intermediate chamber 8a.

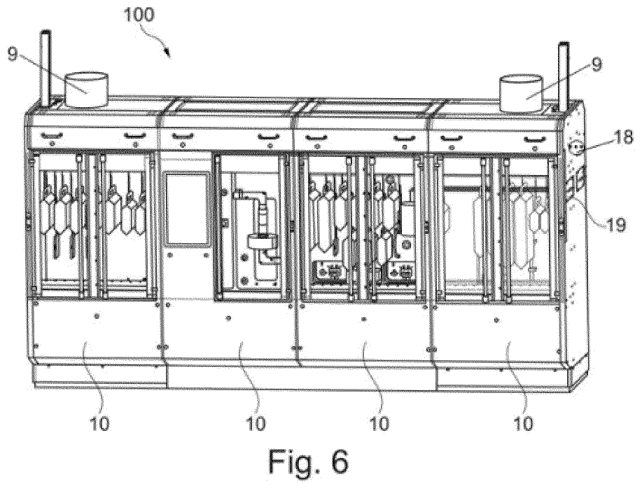

The gas enters the housing 1 before entering the upper intermediate chamber 8a. The gas exits the housing 1 out of the upper intermediate chamber 8a. The housing 1 therefore has a duct 9 (see FIG. 6) or inlet through the housing 1 for the gas flow from the environment to the intermediate chamber 8a and a duct 9 or outlet through the housing 1 for the gas flow from the intermediate chamber 8a to the environment. The flow unit 20 may supply and/or drain the gas flow by means of the duct 9 into or from the intermediate chamber 8a. The inlet and the outlet are sealed relative to the environment. “Sealed” can be understood as compliant with at least IP64.

The control unit 5 controls the gas flow to provide a positive pressure in the process chamber 2 and a gas flow through the process chamber 2. The gas flow through the process chamber 2 is in form of a gas shower falling in the direction of gravity, which means downwards. The gas of the gas flow is here clean air. The positive pressure is an overpressure relative to the environment outside the housing 1 and the technical chamber 3. The gas shower is essentially laminar. A speed of the gas shower is here in a range of 0.2 to 0.6 m/s. The positive pressure and the gas shower protect the manufacturing of the pharmaceutical product in the process chamber 2 from particulate contamination.

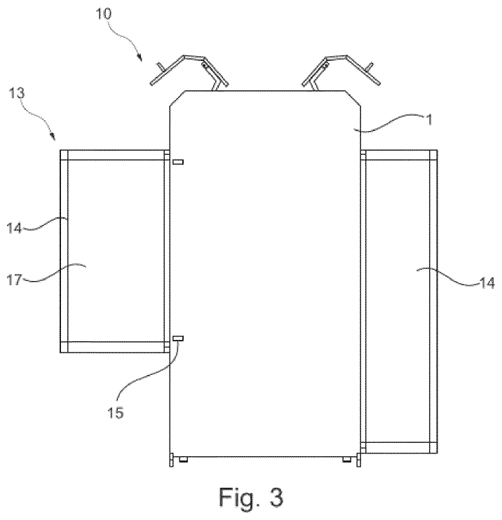

There is a passage for the gas flow from the process chamber 2 to the technical chamber 3. The passage is here via the lower intermediate chamber 8b as part of the technical chamber, which is arranged outside the process chamber 2 and between the process chamber 2 and the technical chamber 3. The passage from the process chamber 2 to the technical chamber 3 is described in more detail with respect to FIG. 4.

The manufacturing device 10 for a pharmaceutical product may be operated under GMP (guidelines for good manufacturing practice)-compliant conditions.

As shown in FIGS. 1 and 2, there is a first filter unit 6 arranged upstream of the process chamber 2 and a second filter unit 7 arranged between the process chamber 2 and the technical chamber 3. The first filter unit 6 is here a H14 filter. The second filter unit 7 is here a H13 filter. The second filter unit 7 is shown in more detail in FIG. 4.

The control unit 5 controls the gas flow to provide 20 to 120 exchange volumes of gas per hour and a fresh gas supply of at least 20 m³/h per m³ in the process chamber 2. The control unit 5 controls the gas flow to provide in the technical chamber 3 a gas exhaust of at least 20 m³/h per m³ technical chamber 3. Preferably, the control unit 5 controls the gas flow to provide a gas exhaust out of the technical chamber 3, which is higher than the fresh gas supply into the process chamber 2. The gas exhaust out of the technical chamber 3 may then be 50 to 200 m³/h.

The control unit 5 controls the gas flow to provide a pressure difference between the process chamber 2 and the technical chamber 3 in a range of 5 to 100 Pa. This means there is a higher pressure in the process chamber 2 than in the technical chamber 3. There may be even a negative pressure in the technical chamber 3 relative to the environment.

As shown in FIGS. 1 and 2, the technical chamber 3 is larger than the process chamber 2 and therefore provides a larger available space to the gas compared to the process chamber 2. The gas in the technical chamber 3 expands relative to the process chamber 2 and the pressure in the technical chamber 3 is lower than in the pressure in the process chamber 2. The larger available space in the technical chamber 3 may suck the gas from the process chamber 2 to the technical chamber 3. Further, the expansion of the gas in the technical chamber 3 may lead to a decrease of temperature of the gas flow. This may be beneficial to cool the apparatus arranged in the technical chamber 3, as e.g. a processor, a motor, a mixer, a pump or the like.

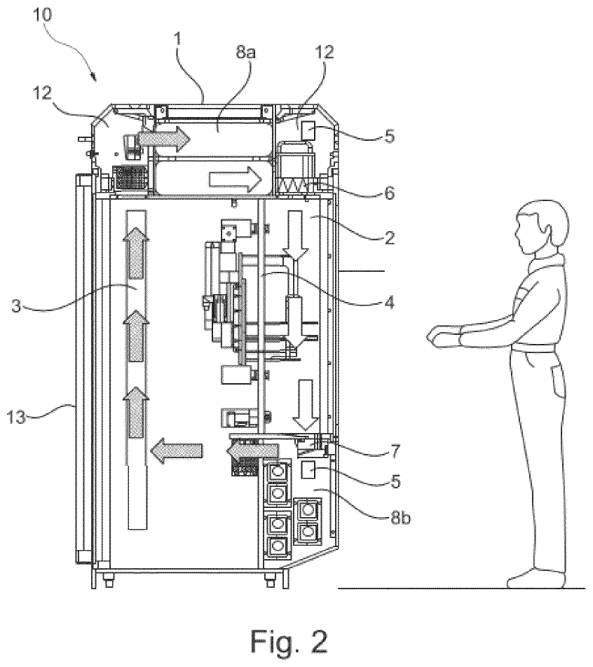

The housing 1 further comprises a control cabinet 12 for the process chamber 2 and a control cabinet 12 for the technical chamber 3. The control cabinets 12 are arranged outside the process chamber 2 and the technical chamber 3. The control cabinets 12 comprise control components for the process chamber 2 and the technical chamber 3, respectively. As shown in FIG. 3, the control cabinets 12 are openable by gullwing doors.

FIG. 3 shows a side view of a manufacturing device 10 for a pharmaceutical product according to the invention. The housing 1 comprises a door unit 13 in the process chamber 2 and a door unit 13 in the technical chamber 3. The door units 13 are openable by means of swing doors to allow access into the respective chamber. The door units 13 therefore each comprise a door 14 and a door frame. The doors 14 are sealed relative to the respective door frame in a closed condition of the door 14. The seal is here a silicone frame. “Sealed” can be understood as compliant with at least IP64. The door units 13 comprise door hinges 15 arranged between the door 14 and the door frame. The door hinges 15 are embedded into the door 14 and the door frame. The door units 13 here comprises a window 17 directed into the process chamber 2.

FIG. 4 shows a detail of a manufacturing device 10 for a pharmaceutical product according to the invention. The detail shows the passage for the gas flow from the process chamber 2 to the technical chamber 3. The passage extends through the intermediate chamber as part of the technical chamber between the process chamber 2 and the technical chamber 3. There is the second filter unit 7 shown, which is arranged between the process chamber 2 and the technical chamber 3. The second filter unit 7 is a barrier, here in form of a H13 filter. There is a collect tray 11 arranged at the passage from the process chamber 2 to the technical chamber 3 to collect liquid droplets.

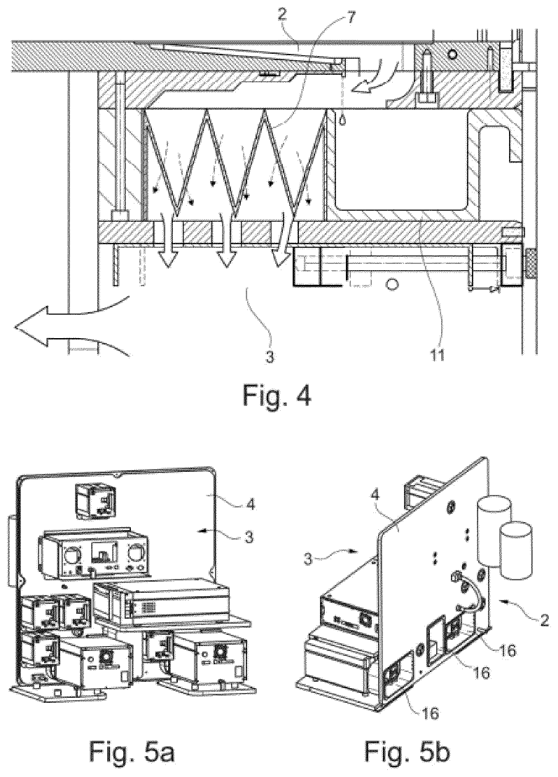

This is beneficial as liquid can be collected before harming the apparatus arranged in the technical chamber 3. FIGS. 5a and 5b show three-dimensional views of the separation element 4. The separation element 4 separates the process chamber 2 from the technical chamber 3. The separation element 4 has a plate-shape and is used to mount apparatuses thereto, here from both sides. The mounted apparatus can be encapsulated or covered to reduce a turbulence or disturbance of the gas flow, prevent precipitation and/or ease a cleaning.

The separation element 4 comprises several appendixes 16 with respective openings towards the process chamber 2 and respective extensions in the direction of the technical chamber 3. The appendixes 16 are used to house apparatuses, as e.g. a pump, a motor, a mixer, a processor or the like. This allows increasing the available space in the process chamber 2, the gas flow is not disturbed and precipitation surfaces are reduced. The appendixes 16 may be closed by covers.

FIG. 6 shows schematically and exemplarily a manufacturing module 100 for a pharmaceutical product according to the invention. The manufacturing module 100 comprises here four manufacturing devices 10 as described above.

The manufacturing device 10 is very flexible and several manufacturing devices 10 can be adapted to several manufacturing steps to form e.g. a manufacturing chain.

The four manufacturing devices 10 are coupled with each other by means of a coupling unit 18 arranged at an outer wall of the housing 1. The coupling unit 18 allows a fluid communication and coupling, and preferably also a mechanical coupling as well as a data communication and coupling of the manufacturing devices 10. The four manufacturing devices 10 are exemplary shown herein to be (from the left) a first device 10 comprising a process media supply unit and a mixing unit, a second device 10 comprising a bioreactor for RNA in vitro transcription, a third device 10 comprising a purification unit and a fourth device 10 comprising a filtration unit, wherein the filtration unit comprises two filtration units, namely a tangential flow filtration unit and a sterile filter. This exemplary module can be used for the production of RNA.

The manufacturing device 10 or module comprises an attachment element 19 arranged at an outer wall of the housing 1 of the manufacturing device 10 outside the housing 1. The attachment element 19 is configured to hold a media supply (not shown) e.g. in form of a media reservoir.

### Definitions

For the sake of clarity and readability the following definitions are provided. Any technical feature mentioned for these definitions may be read on each and every embodiment of the invention. Additional definitions and explanations may be specifically provided in the context of these embodiments.

As used in the specification and the claims, the singular forms of “a” and “an” also include the corresponding plurals unless the context clearly dictates otherwise.

The term “about” in the context of the present invention denotes an interval of accuracy that a person skilled in the art will understand to still ensure the technical effect of the feature in question. The term typically indicates a deviation from the indicated numerical value of ±10% and preferably f5%.

It needs to be understood that the term “comprising” is not limiting. For the purposes of the present invention, the term “consisting of” is considered to be a preferred embodiment of the term “comprising of”. If hereinafter a group is defined to comprise at least a certain number of embodiments, this is also meant to encompass a group which preferably consists of these embodiments only.

The term “pharmaceutical product” as used herein relates to an active pharmaceutical ingredient or any precursor or any intermediate thereof. Thus, a “pharmaceutical product” may in particular be the active pharmaceutical ingredient that is used in a medicament and administered to a human or animal subject in order to treat or prevent a disease, i.e. it has a clinical grade, particularly when it comes to parameters such as purity, integrity. Such products are typically produced in vitro in a synthetic process, and the process of production includes precursors and intermediates. For the present invention, it is particularly preferred that the active pharmaceutical ingredient is a biomolecule, in particular a peptide, a protein or a nucleic acid. The nucleic acid may be DNA or RNA, wherein it is preferred that the nucleic acid is RNA, even more preferably mRNA. The mRNA may particularly be used as a vaccine. When producing e.g. an mRNA as the active pharmaceutical ingredient, a first step may be the production of template DNA (also referred to as “a DNA template” herein), wherein this template DNA corresponds to a precursor of the mRNA since it serves as template in the in vitro transcription reaction when producing the RNA.

An intermediate of the mRNA production may be the mRNA that is obtained from the in vitro transcription reaction before purification since such an mRNA does not correspond to the final active pharmaceutical ingredient but inter alia requires that purification steps are carried out in order to provide the product in the required clinical grade.

The term “DNA” is the usual abbreviation for deoxyribonucleic acid. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers or analogs thereof which are—by themselves—composed of a sugar moiety (deoxyribose), a base moiety and a phosphate moiety, and polymerize by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA-sequence. DNA may be single stranded or double stranded. In the double stranded form, the nucleotides of the first strand typically hybridize with the nucleotides of the second strand, e.g. by A/T-base-pairing and G/C-base-pairing.

The term “RNA” is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. RNA can be obtained by transcription of a DNA sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, a coding sequence, optionally a 3′UTR and a poly(A) sequence. If RNA molecules are of synthetic origin, as in the present invention, the RNA molecules are meant not to be produced in vivo, i.e. inside a cell or purified from a cell, but in an in vitro method. An examples for a suitable in vitro method is in vitro transcription. In addition to messenger RNA, several non-coding types of RNA exist which may be involved in regulation of transcription and/or translation, and immunostimulation and which may also be produced by in vitrotranscription. The term “RNA” further encompasses RNA molecules, such as viral RNA, retroviral RNA and replicon RNA, small interfering RNA (siRNA), antisense RNA, saRNA (small activating RNA), CRISPR RNA (small guide RNA, sgRNA), ribozymes, aptamers, riboswitches, immunostimulating RNA, transfer RNA (tRNA), ribosomal RNA (rRNA), small nuclear RNA (snRNA), small nucleolar RNA (snoRNA), microRNA (miRNA), and Piwi-interacting RNA (piRNA).

The term “RNA in vitro transcription” relates to a process wherein RNA is synthesized in a cell-free system. RNA may be obtained by DNA-dependent RNA in vitro transcription of an appropriate DNA template, which may be a linearized plasmid DNA template or a PCR-amplified DNA template. The promoter for controlling RNA in vitro transcription can be any promoter for any DNA-dependent RNA polymerase. Particular examples of DNA-dependent RNA polymerases are the T7, T3, SP6, or Syn5 RNA polymerases.

Reagents used in RNA in vitro transcription typically include: a DNA template (linearized DNA or linear PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue (e.g. m7G(5′)ppp(5′)G (m7G) or a cap analogue derivable from the structure disclosed in claim 1-5 of WO2017/053297 or any cap structures derivable from the structure defined in claim 1 or claim 21 of WO2018075827); optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate (inhibitor of RNA synthesis); MgCl2, which supplies Mg2+ ions as a co-factor for the polymerase; a buffer (TRIS or HEPES) to maintain a suitable pH value, which can also contain antioxidants (e.g. DTT), and/or polyamines such as spermidine at optimal concentrations, e.g. a buffer system comprising Citrate and/or betaine as disclosed in WO2017/109161.

The nucleotide mixture used in RNA in vitro transcription may additionally contain modified nucleotides as defined herein. In that context, preferred modified nucleotides comprise pseudouridine (Ψ), N1—methylpseudouridine (m1Ω), 5-methylcytosine, and 5-methoxyuridine. The nucleotide mixture (i.e. the fraction of each nucleotide in the mixture) used for RNA in vitro transcription reactions may be optimized for the given RNA sequence, preferably as described in WO2015188933.

An “RNA in vitro transcription (IVT) master mix” may comprise the components necessary for performing an RNA in vitro transcription reaction as defined above. Accordingly, an IVT master mix may comprise at least one of the components selected from a nucleotide mixture, a cap analogue, a DNA-dependent RNA polymerase, an RNAse inhibitor, a pyrophosphatase, MgCl2, a buffer, an antioxidant, betaine, Citrate.

As used herein, the term “template DNA” (or “a DNA template”) typically relates to a DNA molecule comprising a nucleic acid sequence encoding the RNA sequence to be transcribed in vitro. The template DNA is used as template for RNA in vitro transcription in order to produce the RNA encoded by the template DNA. Therefore, the template DNA comprises all elements necessary for RNA in vitro transcription, particularly a promoter element for binding of a DNA dependent RNA polymerase as e.g. T3, T7 and SP6 RNA polymerases 5′ of the DNA sequence encoding the target RNA sequence. Furthermore, the template DNA may comprise primer binding sites 5′ and/or 3′ of the DNA sequence encoding the target RNA sequence to determine the presence of the DNA sequence encoding the target RNA sequence e.g. by PCR or DNA sequencing.

The term “Doggybone™” (dbDNA) as used herein denotes a minimal, closed-linear DNA vector enzymatically developed by Touchlight Genetics Ltd. The linear dbDNA is rapidly produced, plasmid-free and synthesized through an enzymatic process that yields a vector cassette containing only the encoded sequence of interest, promoter, e.g. poly A tail and telomeric ends.

The “polymerase chain reaction” or “PCR” is a technology in molecular biology used to amplify a piece of DNA across several orders of magnitude, generating thousands to millions of copies of a particular DNA sequence. The method relies on thermal cycling, consisting of cycles of repeated heating and cooling of the reaction for DNA melting and enzymatic replication of the DNA. Primers (short DNA fragments) containing sequences complementary to the target sequence along with a heat-stable DNA polymerase, such as Taq polymerase, enable selective and repeated amplification. As PCR progresses, the DNA generated is itself used as a template for replication, setting in motion a chain reaction in which the DNA template is exponentially amplified. The DNA polymerase enzymatically assembles a new DNA strand from DNA building-blocks, the nucleotides, by using single-stranded DNA as a PCR template and DNA oligonucleotides (also called DNA primers), which are required for initiation of DNA synthesis. The vast majority of PCR methods use thermal cycling, i.e., alternately heating and cooling the PCR sample through a defined series of temperature steps. In the first step, the two strands of the DNA double helix are physically separated at a high temperature in a process called DNA melting. In the second step, the temperature is lowered and the two DNA strands become templates for DNA polymerase to selectively amplify the target DNA. The selectivity of PCR results from the use of primers that are complementary to the DNA region targeted for amplification under specific thermal cycling conditions.

An “PCR master mix” may comprise the components necessary for performing a PCR as defined above. Accordingly, a PCR master mix may comprise at least one of the components selected from a nucleotide mixture, a DNA polymerase, the synthetic DNA as (initial) template and a buffer.

The term “lipid nanoparticle” or “LNP” refers to a formulation of the pharmaceutical product, in particular the RNA. In the context of the present invention, the term “LNP” is not restricted to any particular morphology, and includes any morphology generated when a cationic lipid and optionally one or more further lipids are combined, e.g. in an aqueous environment and/or in the presence of an RNA. For example, a liposome, a lipid complex, a lipoplex and the like are within the scope of a lipid nanoparticle (LNP). LNPs typically comprise a cationic lipid and one or more excipients selected from neutral lipids, charged lipids, steroids and polymer conjugated lipids (e.g., PEGylated lipid).

The RNA may be encapsulated in the lipid portion of the LNP or an aqueous space enveloped by some or the entire lipid portion of the LNP. The RNA or a portion thereof may also be associated and complexed with the LNP. An LNP may comprise any lipid capable of forming a particle to which the one or more RNA molecules are attached, or in which the one or more RNA molecules are encapsulated. Preferably, the LNP comprising one or more RNA molecules comprises one or more cationic lipids, and one or more stabilizing lipids. Stabilizing lipids include neutral lipids and PEGylated lipids. In one embodiment, the LNP consists essentially of (i) at least one cationic lipid; (ii) a neutral lipid; (iii) a sterol, e.g. cholesterol; and (iv) a PEG-lipid, e.g. PEG-DMG or PEG-cDMA, in a molar ratio of about 20-60% cationic lipid: 5-25% neutral lipid: 25-55% sterol; 0.5-15% PEG-lipid.

The term “process medium” refers to a component that is directly and physically involved in any reaction or any process step required to produce the pharmaceutical product. Accordingly, as the production is carried out in the process chamber, a “process medium” will be present in the process chamber when the device is used for production. A process medium can thus be in particular any starting material (such as e.g. a nucleotide), any catalyzing material (such as e.g. an enzyme) and any buffer (such as e.g. a reaction buffer or a purification buffer). A process medium is typically provided in a process medium supply container.

The term “technical medium” refers to a component that is not directly involved in any reaction or any process step required to produce the pharmaceutical product. Rather, a technical medium is indirectly involved, e.g. as a power cable that provides power to a unit or apparatus that processes the process medium, and will be located in the technical chamber. A technical medium supply will e.g. be power or power supply provided by a power cable.

It has to be noted that embodiments of the invention are described with reference to different subject matters. In particular, some embodiments are described with reference to method type claims whereas other embodiments are described with reference to the device type claims. However, a person skilled in the art will gather from the description that, unless otherwise notified, in addition to any combination of features belonging to one type of subject matter also any combination between features relating to different subject matters is considered to be disclosed with this application. However, all features can be combined providing synergetic effects that are more than the simple summation of the features.

If a group is defined to comprise at least a certain number of embodiments, this is also meant to encompass a group, which preferably consists of these embodiments only.

While the invention has been illustrated and described in detail in the drawings and the description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. The mere fact that certain measures are re-cited in mutually different dependent claims does not indicate that a combination of these measures cannot be used to advantage. Any reference signs in the claims should not be construed as limiting the scope.

### LIST OF REFERENCES SIGNS

1 housing
2 process chamber
3 technical chamber
4 separation element
5 control unit
6 first filter unit
7 second filter unit
8 upper intermediate chamber
8 lower intermediate chamber
9 duct
10 manufacturing device
11 collect tray
12 control cabinet
13 door unit
14 door
door hinge
16 appendix
17 window
18 coupling unit
19 attachment element
flow unit
100 manufacturing module
   Preferred embodiments of the present application relate to:

1. A manufacturing device (10) for a pharmaceutical product, comprising:
a housing (1),
a process chamber (2),
a technical chamber (3),
a separation element (4), and
a control unit (5),
wherein the housing (1) is closed relative to the environment outside the housing (1),
wherein the housing (1) encompasses the process chamber (2) and the technical chamber (3),
wherein the process chamber (2) is separated from the technical chamber (3) by the separation element (4),
wherein the control unit (5) is configured to control a gas flow through the process chamber (2),
wherein the control unit (5) is configured to control the gas flow to provide in the process chamber (2) a positive pressure relative to the environment, and
wherein the control unit (5) is further configured to control the gas flow to provide the gas flow through the process chamber (2) as a gas shower falling in the direction of gravity. 2. A manufacturing device (10) for a pharmaceutical product, comprising:
a housing (1),
a process chamber (2),
a technical chamber (3),
a separation element (4), and
a control unit (5),
wherein the housing (1) is closed relative to the environment outside the housing (1),
wherein the housing (1) encompasses the process chamber (2) and the technical chamber (3),
wherein the process chamber (2) is separated from the technical chamber (3) by the separation element (4),
wherein the control unit (5) is configured to control a gas flow through the process chamber (2),
wherein the control unit (5) is configured to control the gas flow to provide in the process chamber (2) a positive pressure relative to the environment,
wherein the control unit (5) is further configured to control the gas flow to provide the gas flow through the process chamber (2) as a gas shower falling in the direction of gravity, further comprising a process media supply unit, a mixing unit, a DNA template generation unit, a purification unit, a filtration unit, and an RNA generation unit, wherein each unit comprises (i) a technical media supply, wherein the technical media supply is located in the technical chamber (3), (ii) a process media supply and/or a device configured to be in contact with the process media, wherein the process media supply and/or the device are located in the process chamber (2), and (iii) a sealed through hole located in the separation element (4) and configured to connect the technical media supply and the device configured to be in contact with the process media.

3. A manufacturing device (10) for a pharmaceutical product, comprising:
a housing (1),
a process chamber (2),
a technical chamber (3),
a separation element (4), and
a control unit (5),
wherein the housing (1) is closed relative to the environment outside the housing (1),
wherein the housing (1) encompasses the process chamber (2) and the technical chamber (3),
wherein the process chamber (2) is separated from the technical chamber (3) by the separation element (4),
wherein the control unit (5) is configured to control a gas flow through the process chamber (2),
wherein the control unit (5) is configured to control the gas flow to provide in the process chamber (2) a positive pressure relative to the environment,
wherein the control unit (5) is further configured to control the gas flow to provide the gas flow through the process chamber (2) as a gas shower falling in the direction of gravity,
wherein the technical chamber is configured to hold a technical media supply,
wherein the process chamber is suitable for manufacturing the pharmaceutical product and configured to hold a process media supply and a device configured to be in contact with the process media, and
wherein the separation element is plate shaped and comprises a sealed through hole configured to connect the technical media supply and the device configured to be in contact with the process media.
4. A manufacturing device (10) for a pharmaceutical product, comprising:
a housing (1),
a process chamber (2),
a technical chamber (3),
a separation element (4), and
a control unit (5),
wherein the housing (1) is closed relative to the environment outside the housing (1),
wherein the housing (1) encompasses the process chamber (2) and the technical chamber (3),
wherein the process chamber (2) is separated from the technical chamber (3) by the separation element (4),
wherein the control unit (5) is configured to control a gas flow through the process chamber (2),
wherein the control unit (5) is configured to control the gas flow to provide in the process chamber (2) a positive pressure relative to the environment, wherein the control unit (5) is further configured to control the gas flow to provide the gas flow through the process chamber (2) as a gas shower falling in the direction of gravity, wherein the process chamber is suitable and used for manufacturing the pharmaceutical product; wherein the technical chamber is suitable and used for housing apparatus and technical media supply; and wherein the technical chamber (3) is dimensioned to provide a larger available space to the gas compared to the process chamber(2) to expand the gas in the technical chamber (3) to provide a lower pressure compared to the process chamber (2).

5. The manufacturing device (10) according to any one of embodiments 1 to 4, wherein at least the process chamber (2), preferably the manufacturing device (10), is operable according to GMP requirements.

6. The manufacturing device (10) according to embodiment 5, wherein the GMP requirements are the requirements of the EU guidelines for good manufacturing practice for medicinal products, annex 1.

7. The manufacturing device (10) according to embodiment 5, wherein the GMP requirements are the requirements of the FDA and/or cGMP.

8. The manufacturing device (10) according to one of the preceding embodiments, wherein the process chamber (2) is configured to be at least a grade D room according to the EU guidelines for good manufacturing practice for medicinal products, annex 1, preferably at least a grade C room, more preferably at least a grade B room, even more preferably a grade A room.

9. The manufacturing device (10) according to one of the preceding embodiments, wherein the gas shower is laminar.

10. The manufacturing device (10) according to one of the preceding embodiments, wherein a speed of the gas shower is in a range of about 0.2 to about 0.6 m/s, preferably about 0.36 to about 0.54 m/s.

11. The manufacturing device (10) according to one of the preceding embodiments, further comprising a first filter unit (6) arranged upstream of the process chamber (2), wherein the first filter unit (6) comprises at least a H14 filter.

12. The manufacturing device (10) according to one of the preceding embodiments, further comprising a second filter unit (7) arranged between the process chamber (2) and the technical chamber (3), wherein the second filter unit (7) comprises at least a H13 filter.

13. The manufacturing device (10) according to one of the preceding embodiments, wherein the control unit (5) is configured to control the gas flow to provide in the process chamber about 20 to about 120 exchange volumes of gas per hour and per m³ process chamber, preferably about 50 to about 100 exchange volumes of gas per hour and per m³ process chamber.

14. The manufacturing device (10) according to one of the preceding embodiments, wherein the control unit (5) is configured to control the gas flow to provide in the process chamber (2) a fresh gas supply of about 50 to about 200 m³/h, preferably about 75 to about 150 m³/h.

15. The manufacturing device (10) according to one of the preceding embodiments, wherein the control unit (5) is configured to control the gas flow to provide a gas flow from the process chamber (2) into the technical chamber (3).

16. The manufacturing device (10) according to one of the preceding embodiments, wherein the housing (1) comprises a passage for the gas flow from the process chamber (2) to the technical chamber (3).

17. The manufacturing device (10) according to embodiment 16, wherein the passage transvers the separation element (4).

18. The manufacturing device (10) according to embodiment 16, wherein the passage transvers an intermediate chamber (8) arranged outside the process chamber (2) and the technical chamber (3).

19. The manufacturing device (10) according to one of the embodiments 16 to 18, wherein a valve is arranged in the passage to control the gas flow.

20. The manufacturing device (10) according to one of the preceding embodiments, wherein the control unit (5) is configured to control the gas flow to provide the gas flow in the technical chamber (3) parallel to the gas flow in the process chamber (2), but in an opposite direction.

21. The manufacturing device (10) according to one of the preceding embodiments, wherein the control unit (5) is configured to control the gas flow to provide a pressure difference between the process chamber (2) and the technical chamber (3) in a range of about 5 to about 100 Pa, preferably about 10 to about 15 Pa.

22. The manufacturing device (10) according to one of the preceding embodiments, wherein the control unit (5) is configured to control the gas flow to provide in the technical chamber (3) a gas exhaust of about 200 to about 500 m³/h, preferably about 300 to about 600 m³/h per m³ technical chamber (3).

23. The manufacturing device (10) according to one of the preceding embodiments, further comprising at least a duct (9) through the housing (1) for a passage of the gas flow between the housing (1) and the environment, wherein the duct (9) is sealed relative to the environment.

24. The manufacturing device (10) according to one of the preceding embodiments, wherein the technical chamber (3) is dimensioned to provide a larger available space to the gas compared to the process chamber (2) to expand the gas in the technical chamber (3) to provide a lower pressure compared to the process chamber (2).

25. The manufacturing device (10) according to one of the preceding embodiments, wherein the technical chamber (3) is dimensioned to provide a larger available space to the gas compared to the process chamber (2) to suck the gas from the process chamber (2) to the technical chamber (3).

26. The manufacturing device (10) according to one of the preceding embodiments, wherein the control unit (5) is configured to control the gas flow to provide in the technical chamber (3) a negative pressure relative to the environment.

27. The manufacturing device (10) according to one of the preceding embodiments, wherein the technical chamber (3) is dimensioned relative to the process chamber (2) to expand the gas flow from the process chamber (2) to cool the gas flow in the technical chamber (3) relative to the process chamber (2).

28. The manufacturing device (10) according to one of the preceding embodiments, further comprising a collect tray (11) arranged at the passage from the process chamber (2) to the technical chamber (3) to collect liquid droplets.

29. The manufacturing device (10) according to one of the preceding embodiments, further comprising a cooling unit configured to cool the gas flow in the technical chamber (3) relative to the environment and/or the process chamber (2).

30. The manufacturing device (10) according to one of the preceding embodiments, further comprising a heating unit configured to heat the gas flow in the process chamber (2) relative to the environment.

31. The manufacturing device (10) according to one of the preceding embodiments, further comprising a humidity unit arranged at the passage from the process chamber (2) to the technical chamber (3) to control a humidity of the gas flow.

32. The manufacturing device (10) according to one of the preceding embodiments, further comprising a sensor unit comprising at least one of a group of a flow sensor, a pressure sensor, a temperature sensor, a humidity sensor and a leakage sensor.

33. The manufacturing device (10) according to one of the preceding embodiments, wherein the housing (1) comprises at least an openable control cabinet (12) arranged outside the process chamber (2) and the technical chamber (3), wherein the control cabinet (12) comprises a control component for the process chamber (2) and/or the technical chamber (3).

34. The manufacturing device (10) according to one of the preceding embodiments, wherein the housing (1) comprises an openable door unit (13) in the process chamber (2) and/or in the technical chamber (3), wherein the door unit (13) comprises a door (14) and a door frame, and wherein the door (14) is sealed relative to the door frame in a closed condition of the door (14).

35. The manufacturing device (10) according to the preceding embodiment, wherein the door unit (13) comprises a door hinge (15) arranged between the door (14) and the door frame, wherein the door hinge (15) is preferably at least partially embedded into the door (14) and/or the door frame.

36. The manufacturing device (10) according to one of the embodiments 34 or 35, wherein the door unit (13) comprises a window (17) directed into the process chamber (2) and/or in the technical chamber (3).

37. The manufacturing device (10) according to one of the preceding embodiments, wherein the separation element (4) is plate shaped with an appendix (16) with an opening towards the process chamber (2) and protruding in the direction of the technical chamber (3).

38. The manufacturing device (10) according to one of the preceding embodiments, wherein the housing (1) and/or the separation element (4) is made of aluminium.

39. The manufacturing device (10) according to one of the preceding embodiments, wherein outer surfaces of the housing (1) have at least partially an anti-microbial surface finish.

40. The manufacturing device (10) according to one of the preceding embodiments, further comprising an additive unit arranged within the housing (1) and configured to add an additive to the gas flow.

41. The manufacturing device (10) according to one of the preceding embodiments, further comprising a coupling unit (18) arranged at the housing (1) and configured to couple the manufacturing device (10) to another manufacturing device (10).

42. The manufacturing device (10) according to one of the preceding embodiments, further comprising at least one of a group comprising a process media supply unit, a mixing unit, a DNA template generation unit, an RNA generation unit, a purification unit, a filtration unit, a fill-and-finish unit, and combinations thereof.

43. The manufacturing device (10) according to one of embodiments 1 to 41, further comprising a process media supply unit, a mixing unit, a DNA template generation unit, a purification unit and a filtration unit, wherein the units are preferably connected in the order as listed and wherein the device is preferably configured to produce DNA, preferably template DNA.

44. The manufacturing device (10) according to one of embodiments 1 to 41, further comprising a process media supply unit, a mixing unit, an RNA generation unit, a purification unit and a filtration unit, wherein the units are preferably connected in the order as listed and wherein the device is preferably configured to produce RNA, preferably mRNA.

45. The manufacturing device (10) according to one of embodiments 1 to 41, further comprising a process media supply unit, a mixing unit, a purification unit and a filtration unit, wherein the units are preferably arranged in the order as listed and wherein the device is preferably configured to produce formulated RNA, preferably formulated mRNA, more preferably LNP-formulated mRNA.

46. The manufacturing device (10) according to one of embodiments 1 to 41, further comprising a process media supply unit, a mixing unit, a DNA template generation unit, a purification unit, a filtration unit, and an RNA generation unit, wherein the units are preferably arranged in the order of i) a process media supply unit, ii) a mixing unit, iii) a DNA template generation unit, iv) a purification unit, v) a filtration unit, vi) a process media supply unit, vii) a mixing unit, viii) an RNA generation unit, xi) a purification unit, x) a filtration unit, xi) a process media supply unit, xii) a mixing unit, xiii) a purification unit and xiv) a filtration unit and wherein the device is preferably configured to produce formulated RNA, preferably formulated mRNA, more preferably LNP-formulated mRNA.

47. The manufacturing device (10) according to one of embodiments 42 to 46, wherein each unit comprises (i) a technical media supply, wherein the technical media supply is located in the technical chamber (3), (ii) a process media supply and/or a device configured to be in contact with the process media, wherein the process media supply and the device are located in the process chamber (2), and (iii) a sealed through hole located in the separation element (4) and configured to connect the technical media supply and the device configured to be in contact with the process media.

48. The manufacturing device (10) according to one of embodiments 42 to 47, wherein the process media supply unit comprises a mounting element configured to hold a process medium supply container in the process chamber (2).

49. The manufacturing device (10) according to one of embodiments 42 to 47, wherein the mixing unit comprises a mixer as the device configured to be in contact with the process media.

50. The manufacturing device (10) according to one of embodiments 42 to 47, wherein the DNA template generation unit comprises a polymerase chain reaction unit as the device configured to be in contact with the process media.

51. The manufacturing device (10) according to one of embodiments 42 to 47, wherein the RNA generation unit comprises a bioreactor for RNA in vitro transcription as the device configured to be in contact with the process media.

52. The manufacturing device (10) according to one of embodiments 42 to 47, wherein the purification unit comprises at least one chromatography purification unit as the device configured to be in contact with the process media.

53. The manufacturing device (10) according to embodiment 52, wherein the chromatography purification unit comprises a high-pressure liquid chromatography unit and/or an affinity chromatography unit.

54. The manufacturing device (10) according to embodiment 53, wherein the high-pressure liquid chromatography unit is a reversed phase high-pressure liquid chromatography unit, preferably a column, and the affinity chromatography unit is an oligo d(T) affinity chromatography unit, preferably a column.

55. The manufacturing device (10) according to one of embodiments 42 to 47, wherein the filtration unit comprises at least one filter as the device configured to be in contact with the process media.

56. The manufacturing device (10) according to embodiment 55, wherein the filter is a tangential flow filtration unit or a sterile filter, preferably a sterile filter with a size of 0.22 μm.

57. The manufacturing device (10) according to one of embodiments 42 to 47, wherein the fill-and-finish unit comprises a filling and/or dosing machine as the device configured to be in contact with the process media.

58. A manufacturing module (100) for a pharmaceutical product, comprising at least two manufacturing devices (10) according to one of embodiments 1 to 57, wherein the at least two manufacturing devices (10) are coupled with each other.

59. The manufacturing module (100) according to embodiment 58, wherein (i) a first manufacturing device comprises a process media supply unit and a mixing unit, (ii) a second manufacturing device comprises a DNA template generation unit, (iii) a third manufacturing device comprises a purification unit and (iv) a fourth manufacturing device comprises a filtration unit.

60. The manufacturing module (100) according to embodiment 59, further configured to produce DNA, preferably template DNA.

61. The manufacturing module (100) according to embodiment 58, wherein (i) a first manufacturing device comprises a process media supply unit and a mixing unit, (ii) a second manufacturing device comprises a bioreactor for RNA in vitro transcription, (iii) a third manufacturing device comprises a purification unit and (iv) a fourth manufacturing device comprises a filtration unit, wherein the fourth device preferably comprises two filtration units, namely a tangential flow filtration unit and a sterile filter.

62. The manufacturing module (100) according to embodiment 61, further configured to produce RNA, preferably mRNA.

63. The manufacturing module (100) according to embodiment 58, wherein (i) a first manufacturing device comprises a process media supply unit and a mixing unit, (ii) a second manufacturing device comprises a filtration unit, preferably a tangential flow filtration unit, and (iii) a third manufacturing device comprises a filtration unit, preferably a sterile filter.

64. The manufacturing module (100) according to embodiment 63, further configured to produce formulated RNA, preferably formulated mRNA, more preferably LNP-formulated mRNA.

65. The manufacturing module (100) according to embodiment 58, wherein a first manufacturing device comprises a process media supply unit and a mixing unit, (ii) a second manufacturing device comprises a DNA template generation unit, (iii) a third manufacturing device comprises a purification unit, (iv) a fourth manufacturing device comprises a filtration unit, (v) a fifth manufacturing device comprises a process media supply unit and a mixing unit, (vi) a sixth manufacturing device comprises a bioreactor for RNA in vitro transcription, (vii) a seventh manufacturing device comprises a purification unit, (viii) an eight manufacturing device comprises a filtration unit, (ix) a ninth manufacturing device comprises a process media supply unit and a mixing unit, (x) a tenth manufacturing device comprises a filtration unit, preferably a tangential flow filtration unit, and (xi) an eleventh manufacturing device comprises a filtration unit, preferably a sterile filter.

66. The manufacturing module (100) according to embodiment 65, further configured to produce formulated RNA, preferably formulated mRNA, more preferably LNP-formulated mRNA.

67. A manufacturing system for a pharmaceutical product, comprising a manufacturing device (10) according to one of the embodiments 1 to 57 or a manufacturing module (100) according to one of the embodiments 58 to 66 as well as a clean room, wherein the manufacturing device (10) or the manufacturing module (100) is arranged in the clean room.

68. The manufacturing system according to embodiment 67, wherein the clean room is a grade D room according to the EU guidelines for good manufacturing practice for medicinal products, annex 1.

69. The manufacturing system according to embodiment 67 or 68, wherein the clean room is a tent, a cell or a shippable container.

70. A method for producing a pharmaceutical product, wherein the method comprises the following steps:
providing a manufacturing device (10) according to any of embodiments 1 to 57 or a manufacturing module (100) according to any of embodiments 58 to 66 or a manufacturing system according to any of embodiments 67 to 69; wherein the device, module or system comprises a unit for the production of the pharmaceutical product, wherein a technical media supply of the unit is located in a technical chamber (3), a process media supply of the unit and a device configured to be in contact with the process media are located in a process chamber (2), and a separation element (4) comprises a sealed through hole configured to connect the technical media supply and the device of the unit;
providing a gas flow through the process chamber (2), wherein the gas flow through the process chamber (2) is a gas shower falling in the direction of gravity;
providing a positive pressure in the process chamber (2) relative to the environment;
providing a process media and a technical media for the unit; and
operating the unit to obtain the pharmaceutical product,
optionally, sanitizing the process chamber (2), preferably wherein sanitizing is automated.

71. A method for producing a DNA template, wherein the method comprises the following steps:
providing a manufacturing device (10) according to any of embodiments 43 and 47 to 57 or a manufacturing module (100) according to any of embodiments 59 to 60 or a manufacturing system according to any of embodiments 67 to 69;
providing a gas flow through a process chamber (2), wherein the gas flow through the process chamber (2) is a gas shower falling in the direction of gravity;
providing a positive pressure in the process chamber (2) relative to the environment;
providing a process media and a technical media for the units; and
mixing the process media required for a PCR mastermix in a mixing unit, followed by generating a DNA template in a DNA template generation unit, followed by purifying the DNA template in a purification unit and by filtering the DNA template in a filtering unit to obtain the DNA template,
optionally, sanitizing the process chamber (2), preferably wherein sanitizing is automated.

72. A method for producing an RNA, wherein the method comprises the following steps:
providing a manufacturing device (10) according to any of embodiments 44 and 47 to 57 or a manufacturing module (100) according to any of embodiments 61 to 62 or a manufacturing system according to any of embodiments 67 to 69;
providing a gas flow through a process chamber (2), wherein the gas flow through the process chamber (2) is a gas shower falling in the direction of gravity;
providing a positive pressure in the process chamber (2) relative to the environment;
providing a process media and a technical media for the units; and
mixing the process media required for an IVT mastermix in a mixing unit, followed by adding a template DNA and generating an RNA in an RNA generation unit, followed by purifying the RNA in a purification unit and by filtering the RNA in a filtering unit, optionally in two filtering units, namely a tangential flow filtration unit and a sterile filter, to obtain the RNA,
optionally, sanitizing the process chamber (2), preferably wherein sanitizing is automated.

73. A method for producing a formulated RNA, wherein the method comprises the following steps:
providing a manufacturing device (10) according to any of embodiments 45 and 47 to 57 or a manufacturing module (100) according to any of embodiments 63 to 64 or a manufacturing system according to any of embodiments 67 to 69;
providing a gas flow through a process chamber (2), wherein the gas flow through the process chamber (2) is a gas shower falling in the direction of gravity;
providing a positive pressure in the process chamber (2) relative to the environment;
providing a process media and a technical media for the units; and
mixing the process media, preferably a lipid solution, and an RNA in a mixing unit, followed by filtering a formulated RNA in a tangential flow filtration unit and by filtering the formulated RNA in a sterile filter to obtain the formulated RNA,
optionally, sanitizing the process chamber (2), preferably wherein sanitizing is automated.

74. A method for producing a formulated RNA, wherein the method comprises the following steps:
providing a manufacturing device (10) according to any of embodiments 46 to 57 or a manufacturing module (100) according to any of embodiments 65 to 66 or a manufacturing system according to embodiment 67 to 69;
providing a gas flow through a process chamber (2), wherein the gas flow through the process chamber (2) is a gas shower falling in the direction of gravity;
providing a positive pressure in the process chamber (2) relative to the environment;
providing a process media and a technical media for the units; and
mixing the process media required for a PCR mastermix in the mixing unit, followed by generating a DNA template in a DNA template generation unit, followed by purifying the DNA template in a purification unit and by filtering the DNA template in a filtering unit, followed by mixing the process media required for an IVT mastermix in the mixing unit, followed by adding the template DNA and generating an RNA in a RNA generation unit, followed by purifying the RNA in a purification unit and by filtering the RNA in a filtering unit, optionally in two filtering units, namely a tangential flow filtration unit and a sterile filter, followed by mixing the process media, preferably a lipid solution, and the RNA in the mixing unit, followed by filtering a formulated RNA in the tangential flow filtration unit and by filtering the formulated RNA in the sterile filter to obtain the formulated RNA,
optionally, sanitizing the process chamber (2), preferably wherein sanitizing is automated.

75. Use of a manufacturing device (10) according to any of embodiments 1 to 57 or a manufacturing module (100) according to any of embodiments 58 to 66 or a manufacturing system according to any of embodiments 67 to 69, for the production of a pharmaceutical product, preferably an active pharmaceutical ingredient in the form of a biomolecule or any precursor or any intermediate thereof.

76. The use according to embodiment 75, wherein the pharmaceutical product is formulated.

77. The use according to embodiment 75, wherein the biomolecule is a peptide, a protein or a nucleic acid, preferably RNA, more preferably mRNA.

78. Use of a manufacturing device (10) according to any of embodiments 43 and 47 to 57 or a manufacturing module (100) according to any of embodiments 59 to 60 or a manufacturing system according to any of embodiments 67 to 69, for the production of DNA, preferably a DNA template.

79. Use of a manufacturing device (10) according to any of embodiments 44 and 47 to 57 or a manufacturing module (100) according to any of embodiments 61 to 62 or a manufacturing system according to any of embodiments 67 to 69, for the production of RNA, preferably mRNA.

80. Use of a manufacturing device (10) according to any of embodiments 45 to 57, or a manufacturing module (100) according to any of embodiments 63 to 65 or a manufacturing system according to any of embodiments 67 to 69 for the production of formulated RNA, preferably formulated mRNA, more preferably LNP-formulated mRNA.

81. The use according to one of embodiments 75 to 80, wherein the use is automated.

To the Claims:

1. A manufacturing device (10) for a pharmaceutical product, comprising:
a housing (1),
a process chamber (2),
a technical chamber (3),
a separation element (4), and
a control unit (5),
wherein the housing (1) is closed relative to the environment outside the housing (1),
wherein the housing (1) encompasses the process chamber (2) and the technical chamber (3),
wherein the process chamber (2) is separated from the technical chamber (3) by the separation element (4),
wherein the control unit (5) is configured to control a gas flow through the process chamber (2),
wherein the control unit (5) is configured to control the gas flow to provide in the process chamber (2) a positive pressure relative to the environment, and
wherein the control unit (5) is further configured to control the gas flow to provide the gas flow through the process chamber (2) as a gas shower falling in the direction of gravity;
wherein the process chamber (2) is suitable and used for manufacturing the pharmaceutical product;
wherein the technical chamber (3) is suitable and used for providing technical media supply;
wherein the technical chamber (3) is dimensioned to provide a larger available space to the gas compared to the process chamber (2) to expand the gas in the technical chamber (3) to provide a lower pressure compared to the process chamber (2); and
wherein the separation element (4) is plate-shaped.

2. The manufacturing device (10) according to claim 1, wherein at least the process chamber (2), is operable according to GMP requirements.

3. The manufacturing device (10) according to claim 2, wherein the GMP requirements are the requirements of the EU guidelines for good manufacturing practice for medicinal products, annex 1.

4. The manufacturing device (10) according to claim 2, wherein the GMP requirements are the requirements of the FDA and/or cGMP.

5. The manufacturing device (10) according to claim 3, wherein the process chamber (2) is configured to be at least a grade D room according to the EU guidelines for good manufacturing practice for medicinal products, annex 1.

6. The manufacturing device (10) according to claim 1, wherein the gas shower is laminar.

7. The manufacturing device (10) according to claim 1, wherein a speed of the gas shower is in a range of about 0.2 to about 0.6 m/s, or about 0.36 to about 0.54 m/s.

8. The manufacturing device (10) according to claim 1, further comprising a first filter unit (6) arranged upstream of the process chamber (2), wherein the first filter unit (6) comprises at least a H13 filter.

9. The manufacturing device (10) according to claim 8, further comprising a second filter unit (7) arranged between the process chamber (2) and the technical chamber (3), wherein the second filter unit (7) comprises at least a H13 filter.

10. The manufacturing device (10) according to claim 8, wherein the control unit (5) is configured to control the gas flow to provide in the process chamber about 20 to about 120 exchange volumes of gas per hour and per m3 process chamber.

11-16. (canceled)

17. The manufacturing device (10) according claim 8, wherein the control unit (5) is configured to control the gas flow to provide the gas flow from the process chamber (2) into the technical chamber (3) parallel to the gas flow in the process chamber (2), but in an opposite direction.

18-30. (canceled)

31. The manufacturing device (10) according to claim 1, further comprising a sensor unit comprising at least one of a flow sensor, a pressure sensor, a temperature sensor, a humidity sensor, a microbial sensor, a particulate sensor, an organics sensor, and/or a leakage sensor.

32-41. (canceled)

42. The manufacturing device (10) according to claim 1, further comprising a process media supply unit, a mixing unit, a de-novo DNA synthesis unit, a purification unit and a filtration unit, wherein the device is configured to produce DNA.

43-45. (canceled)

46. The manufacturing device (10) according to claim 42, further comprising a process media supply unit, a mixing unit, a de-novo DNA synthesis unit, a DNA template generation unit, a purification unit, a filtration unit, a formulation unit and an RNA generation unit.

47-52. (canceled)

53. The manufacturing device (10) according to claim 46, wherein the purification unit comprises at least one chromatography purification unit as the device configured to be in contact with the process media.

54. The manufacturing device (10) according to claim 53, wherein the chromatography purification unit comprises a high-pressure liquid chromatography unit and/or an affinity chromatography unit.

55-58. (canceled)

59. A manufacturing module (100) for a pharmaceutical product, comprising at least two manufacturing devices (10) according to claim 1, wherein the at least two manufacturing devices (10) are coupled with each other.

60-69. (canceled)

70. A manufacturing system for a pharmaceutical product, comprising a manufacturing device (10) according to claim 1 as well as a clean room, wherein the manufacturing device (10) is arranged in the clean room.

71-72. (canceled)

73. A method for producing a pharmaceutical product, wherein the method comprises the following steps:
providing a manufacturing device (10) according to claim 1;
providing a gas flow through the process chamber (2), wherein the gas flow through the process chamber (2) is a gas shower falling in the direction of gravity;
providing a positive pressure in the process chamber (2) relative to the environment;
providing a process media and a technical media for the unit; and
operating the unit to obtain the pharmaceutical product,
optionally, sanitizing the process chamber (2).

74. A method for producing DNA, wherein the method comprises the following steps:
providing a manufacturing device (10) according to claim 42;
providing a gas flow through a process chamber (2), wherein the gas flow through the process chamber (2) is a gas shower falling in the direction of gravity;
providing a positive pressure in the process chamber (2) relative to the environment;
providing a process media and a technical media for the units; and
mixing the process media required for the de-novo DNA synthesis in a mixing unit, followed by generating DNA in a de-novo DNA synthesis unit, followed by purifying the DNA in a purification unit and by filtering the DNA in a filtering unit to obtain the DNA,
optionally, sanitizing the process chamber (2).

75-86. (canceled)
